(19) United States
(12) Patent Application Publication
(10) Pub. No.: US 2025/0124578 A1
(43) Pub. Date: Apr. 17, 2025

(54) METHOD FOR GENERATING ANEURYSM REGION AND ELECTRONIC DEVICE THEREOF

(71) Applicant: Medipixel, Inc., Seoul (KR)

(72) Inventors: Yeongjun KIM, Seoul (KR); Donghyun WON, Seoul (KR)

(73) Assignee: Medipixel, Inc., Seoul (KR)

(21) Appl. No.: 18/991,923

(22) Filed: Dec. 23, 2024

Related U.S. Application Data
(63) Continuation-in-part of application No. 18/822,855, filed on Sep. 3, 2024.

(30) Foreign Application Priority Data
Sep. 6, 2023 (KR) 10-2023-0118083
Jun. 19, 2024 (KR) 10-2024-0079442

(51) Int. Cl. G06T 7/11; G06T 7/00; G16H 30/40

(52) U.S. Cl. CPC G06T 7/11; G06T 7/0012; G16H 30/40

(57) ABSTRACT

Provided is a method for generating an aneurysm region performed by an electronic device including: obtaining, by at least one processor, input images; generating, by the at least one processor, a vessel mesh based on the input images; determining, by the at least one processor, an adhered vessel from the vessel mesh; and separating, by the at least one processor, the adhered vessel based on user input.

### CROSS-REFERENCE TO RELATED APPLICATIONS

This application is a continuation-in-part of U.S. patent application Ser. No. 18/822,855 filed on Sep. 3, 2024, which claims priority to and the benefit thereof under 35 U.S.C. § 119 to Korean Patent Application No. 10-2023-0118083 filed in the Korean Intellectual Property Office on Sep. 6, 2023, and Korean Patent Application No. 10-2024-0079442 filed in the Korean Intellectual Property Office on Jun. 19, 2024, the entire contents of which are incorporated herein by reference.

### BACKGROUND

### (a) Field

The disclosure relates to a method for generating an aneurysm region and an electronic device thereof.

### (b) Description of the Related Art

An aneurysm is a disease where part of an artery wall weakens and balloons out, which can occur in various forms such as cerebral aneurysm, aortic aneurysm, renal artery aneurysm, and splenic artery aneurysm. Among the treatment methods for aneurysms, coil embolization is a prominent technique where a thin coil is inserted into the aneurysm to block the flow of blood into the cerebral aneurysm.

To perform coil embolization effectively, it is crucial to accurately measure the size of the aneurysm, including volume and length, as these measurements are used to determine the appropriate amount of coil needed. Traditionally, medical professionals estimated the size of the aneurysm by analyzing images and using polyhedral shapes, like octagons, to approximate the size. However, there is an increasing demand for methods that can determine the size more precisely, ensuring it matches the actual shape of the aneurysm.

### SUMMARY

Some embodiments may provide a method for generating an aneurysm region and an electronic device thereof to obtain precise size information of an aneurysm.

According to an aspect of an embodiment, a method for generating an aneurysm region performed by an electronic device may include: generating, by at least one processor, a vessel mesh based on the input images; determining, by the least one processor, an adhered vessel from the vessel mesh; and separating the adhered vessel based on user input.

According to an aspect of an embodiment, an electronic device may include a processor; and a memory connected to the processor, wherein the memory is configured to store a program, the processor is configured to execute the program, and when the program is executed, the steps of a method for generating an aneurysm region are implemented.

Additional aspects may be set forth in part in the description which follows and, in part, may be apparent from the description, and/or may be learned by practice of the presented embodiments.

### BRIEF DESCRIPTION OF THE DRAWINGS

The above and other aspects, features, and advantages of certain embodiments of the present disclosure will become apparent from the following description taken in conjunction with the accompanying drawings, in which:

FIG. 1 is a schematic block diagram of the computing system according to an embodiment.

FIG. 2 is an example flowchart showing a method for generating aneurysm region according to an embodiment.

FIG. 3 is an example flowchart showing a method for generating a vessel network by an electronic device according to an embodiment.

FIGS. 4 to 11 illustrate a method for generating a vessel network by an electronic device according to an embodiment.

FIG. 12 is an example flowchart showing a method for processing a vessel network by an electronic device according to an embodiment.

FIGS. 13 to 18 illustrate a method for processing a vessel network by an electronic device according to an embodiment.

FIG. 19 is an example flowchart showing a method for processing a vessel network by an electronic device according to an embodiment.

FIG. 20 is an example flowchart showing a method for processing a kissing vessel by an electronic device according to an embodiment.

FIGS. 21 to 26 illustrate a method for processing a kissing vessel by an electronic device according to an embodiment.

FIG. 27 illustrates a flowchart of a method for generating a merge strip by an electronic device according to an embodiment.

FIGS. 28 to 35 illustrate a method for generating a merge strip by an electronic device according to an embodiment.

FIG. 36 illustrates a flowchart of a method for separating adhered vessels by an electronic device according to an embodiment.

FIGS. 37 to 44 illustrate a method for separating adhered vessels by an electronic device according to an embodiment.

FIG. 45 illustrates a flowchart of a method for separating adhered vessels by an electronic device according to an embodiment.

FIGS. 46 and 47 illustrate a method for separating adhered vessels by an electronic device according to an embodiment.

### DETAILED DESCRIPTION OF THE INVENTION

In the following detailed description, only certain embodiments of the present invention have been shown and described, simply by way of illustration. As those skilled in the art would realize, the described embodiments may be modified in various different ways, all without departing from the spirit or scope of the present invention.

The drawings and description are to be regarded as illustrative in nature and not restrictive. Like reference numerals designate like elements throughout the specification. The sequence of operations or steps is not limited to the order presented in the claims or figures unless specifically indicated otherwise. The order of operations or steps may be changed, several operations or steps may be merged, a certain operation or step may be divided, and a specific operation or step may not be performed.

As used herein, the singular forms “a” and “an” are intended to include the plural forms as well, unless the context clearly indicates otherwise. Although the terms first, second, and the like may be used herein to describe various elements, components, steps and/or operations, these terms are only used to distinguish one element, component, step or operation from another element, component, step, or operation.

As used herein, each of such phrases as “A or B,” “at least one of A and B,” “at least one of A or B,” “A, B, or C,” “at least one of A, B, and C,” and “at least one of A, B, or C,” may include any possible combination of the items enumerated together in a corresponding one of the phrases.

Reference throughout the present disclosure to “one embodiment,” “an embodiment,” “an example embodiment,” or similar language may indicate that a particular feature, structure, or characteristic described in connection with the indicated embodiment is included in at least one embodiment of the present solution. Thus, the phrases “in one embodiment”, “in an embodiment,” “in an example embodiment,” and similar language throughout this disclosure may, but do not necessarily, all refer to the same embodiment.

Hereinafter, various embodiments of the present disclosure are described with reference to the accompanying drawings.

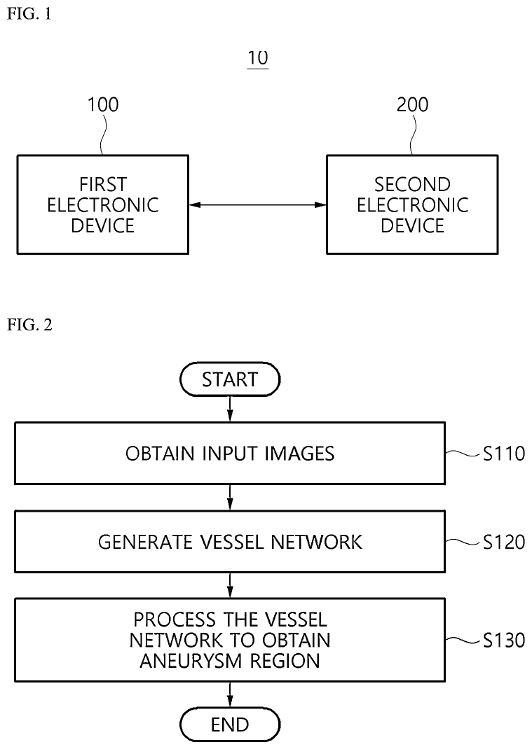

FIG. 1 is a schematic block diagram of the computing system according to an embodiment, and FIG. 2 is an example flowchart showing a method for generating aneurysm region according to an embodiment.

Referring to FIG. 1, a computing system 10 according to an embodiment may obtain a vessel image of a first user, perform image processing on the obtained vessel image, and display the processed image. The computing system 10 may provide the processed image to a second user. For example, the first user may be a patient, and the second user may be a medical professional.

In some embodiments, the computing system 10 may capture a brain vascular of the first user to obtain a brain vascular image and generate an aneurysm region by performing image processing on the brain vascular image. However, the embodiments are not limited thereto, and the computing system 10 may capture blood vessels that are the subject of angiography, such as the cardiovascular system or gastrointestinal vessels.

The computing system 10 according to an embodiment includes a first electronic device 100 and a second electronic device 200. The first electronic device 100 may be an imaging device that captures an image of the first user to obtain an image. For example, the first electronic device 100 may be an angiography equipment (for example, angio-device), Optical Coherence Tomography (OCT) equipment, Computed Tomography (CT) equipment, Magnetic Resonance Imaging (MRI) equipment, or Magnetic Resonance Angiography (MRA) equipment.

The first electronic device 100 may capture the first user from multiple imaging points to obtain the plurality of images. In some embodiments, the first electronic device 100 may capture the first user while rotating around the first user. In other embodiments, the first electronic device 100 may rotate the first user and capture the first user while rotating. The first electronic device 100 may transmit the obtained images to the second electronic device 200.

The second electronic device 200 may generate an aneurysm region based on the plurality of images. The second electronic device 200 may perform a method for generating an aneurysm region shown in FIG. 2. The second electronic device 200 may include a memory that stores a program for executing the method for generating the aneurysm region and a processor configured to execute the program to generate the aneurysm region. The processor may be implemented as one or more processors. The second electronic device 200 may further include input/output devices (such as input devices like a mouse or keyboard, output devices like a display panel, and input/output devices like a touchscreen panel, etc.), communication devices, and others.

Referring to FIG. 1 and FIG. 2, the second electronic device 200 may obtain the input image (S110). For example, the second electronic device 200 may receive the plurality of images captured by the first electronic device 100.

The second electronic device 200 is a computing device that performs image processing on an image input from the first electronic device 100. The second electronic device 200 may generate a vessel network based on the input image (S120).

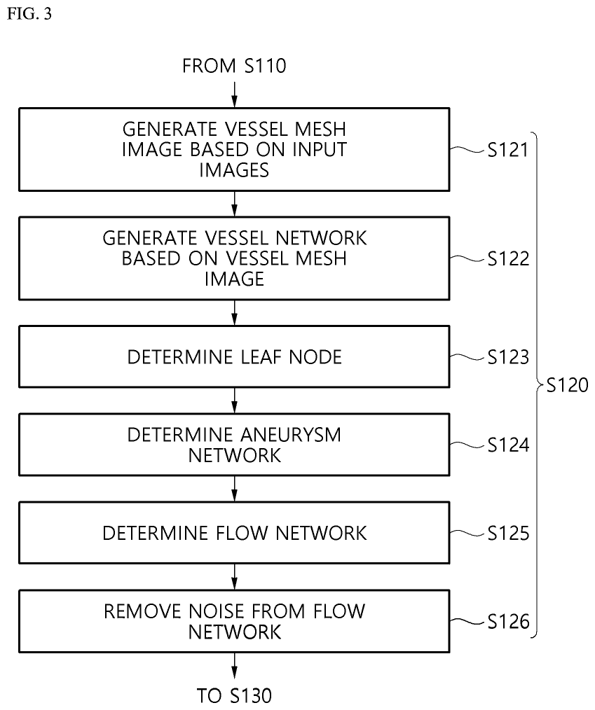

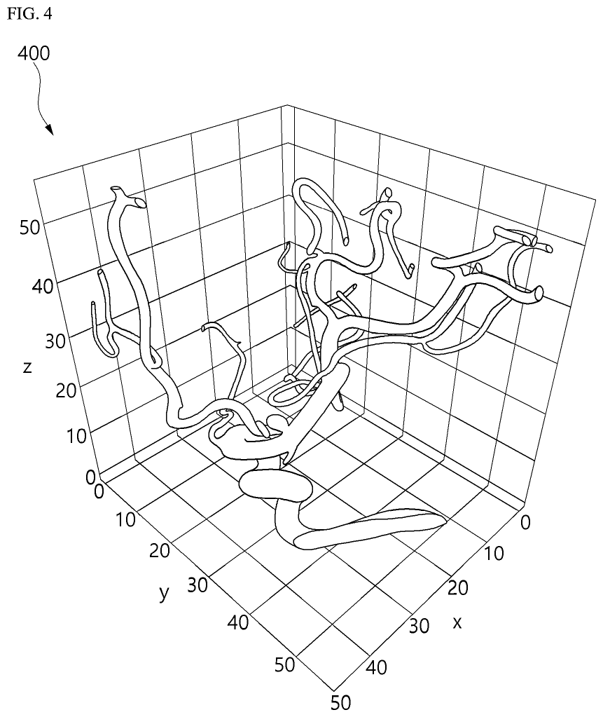

The second electronic device 200 may generate a vessel mesh from the input image. In some embodiments, the second electronic device 200 may generate the vessel mesh using a marching cubes algorithm. The vessel mesh may be data that represents blood vessels in three dimensions (3D). The second electronic device 200 may generate the vessel network based on the vessel mesh. As the vessel mesh does not include 3D parameters such as coordinates or vectors, the second electronic device 200 may generate the vessel network for image processing. The configuration of generating the vessel network by the second electronic device 200 will be described later with reference to FIG. 3.

The second electronic device 200 may process the vessel network to obtain an aneurysm region (S130). The aneurysm region may be an area within the vessel network where the aneurysm is located. In other words, the second electronic device 200 may generate the aneurysm region from the vessel network. Processing the vessel network by the second electronic device 200 may include performing image processing. For example, the second electronic device 200 may select a plurality of nodes within the vessel network and determine a cutting plane based on the selected nodes. The second electronic device 200 may generate the aneurysm region by cutting the vessel network using the cutting plane.

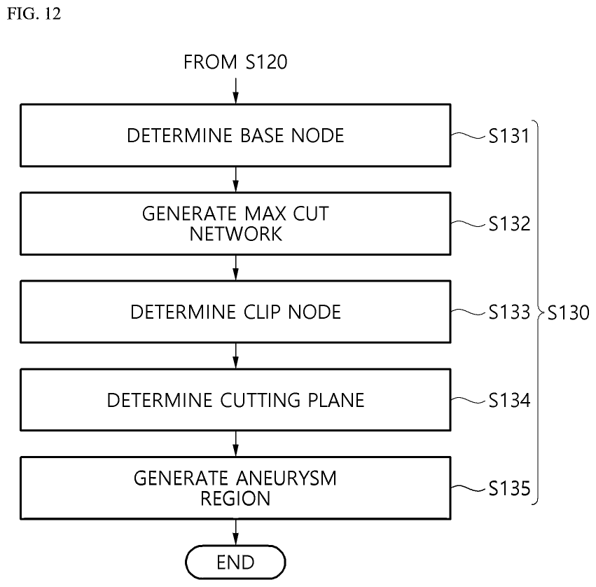

In some embodiments, the second electronic device 200 may generate the aneurysm region by filtering leaf vessels from the vessel network. In some embodiments, the second electronic device 200 may generate the aneurysm region by filtering sticky vessels from the vessel network. In some embodiments, the second electronic device 200 may generate the aneurysm region by filtering noises from the vessel network. The second electronic device 200 may generate the aneurysm region using various combinations of the aforementioned manners. The configuration of processing the vessel network by the electronic device will be described later with reference to FIG. 12.

The second electronic device 200 may determine a point corresponding to the aneurysm region (for example, a clip node). The second electronic device 200 may then determine a cutting plane to cut the vessel image at the determined point. In some embodiments, the second electronic device 200 may verify the clip node and/or the cutting plane. If the verification of the clip node and/or the cutting plane fails, the second electronic device 200 may modify the clip node and/or the cutting plane.

By the second electronic device 200 generating the vessel mesh from the input image and precisely separating the aneurysm region, the second user may precisely measure the parameters of the aneurysm included in the complex vascular structure. Therefore, the second user may make an accurate diagnosis to establish a treatment plan based on the measured parameters.

Also, as the second electronic device 200 is configured to remove noises that may occur during the imaging process, the clarity and accuracy of the vessel mesh may be improved. Accordingly, the necessity for re-imaging may be reduced, treatment time may be shortened, and in the case of performing a (medical) procedure or surgery, the risk may be reduced, thereby enhancing reliability.

The second electronic device 200 may be a server, a data center, an Artificial Intelligence (AI) device, a Personal Computer (PC), a laptop computer, a mobile phone, a smart phone, a tablet PC, a wearable device, a healthcare device, etc.

In some embodiments, if the second electronic device 200 is implemented as a server, data center, etc., the computing system 10 may further include a third electronic device for interacting with the second user. For example, the third electronic device may interact with the second user by displaying images to the second user and receiving input from the second user. The third electronic device may communicate with the first electronic device and/or the second electronic device.

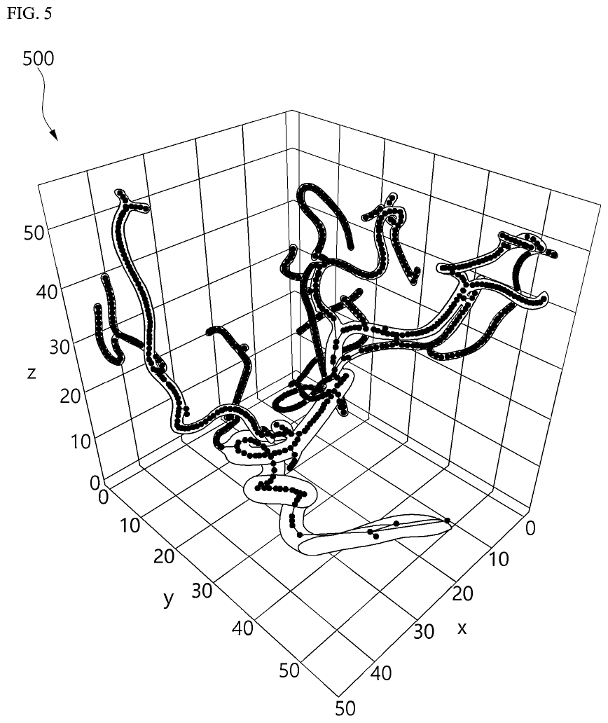

FIG. 3 is an example flowchart showing a method for generating a vessel network by an electronic device according to an embodiment, and FIGS. 4 to 11 illustrate a method for generating a vessel network by an electronic device according to an embodiment.

Referring to FIGS. 3 to 11, the electronic device according to an embodiment (for example, the second electronic device 200 in FIG. 1) may obtain the input image (S110) and generate the vessel mesh 400 based on the input image (S121). The vessel mesh 400 may be as shown in FIG. 4. The vessel mesh 400 may include a three-dimensional surface, and the surface may include a plurality of cells. For example, the cell may be a two-dimensional plane formed using three points.

In some embodiments, the input image may include voxel data, and the electronic device may apply a conversion algorithm to the voxel data to generate the vessel mesh 400. For example, the conversion algorithm may include a marching cubes algorithm.

The electronic device may generate the vessel network 500 based on the vessel mesh 400 (S122). The vessel network 500 may be as shown in FIG. 5.

Generating the vessel network 500 may be understood as generating nodes and edges. A node may refer to a point inside the surface, and an edge may refer to a line that connects two neighboring nodes. The electronic device may generate the nodes by determining the three-dimensional coordinates of the nodes.

The electronic device may detect at least one open vessel in the vessel mesh 400 and determine a boundary of the open vessel. The open vessel may refer to a vessel that is not occluded. The boundary may refer to the cells at the end of the open vessel that are in contact with each other.

The electronic device may generate a start node based on the boundary of the open vessel. For example, the electronic device may generate the center of the cells at the boundary of the open vessel as the start node. The electronic device may start from the surface adjacent to the start node and continue to determine subsequent nodes by performing surface propagation. Surface propagation may refer to moving from the first cells, used to determine the nodes, to the second cells adjacent to the first cells. That is, the electronic device may determine the first node (the start node) from the cells (for example, the first cells) at the boundary of the open vessel and determine the second node from the cells (for example, the second cells) adjacent to the boundary cells. In this manner, the electronic device may generate nodes while performing surface propagation for all the cells on the surface.

The electronic device may determine radius information of the nodes. The radius information may refer to the distance from the node to the surface. For example, the electronic device may calculate the distances between the node and the cells used in determining that node, and determine the radius information based on the distances. In some embodiments, the electronic device may determine the longest distance among the calculated distances as the radius information for that node. However, the embodiments are not limited thereto, and the electronic device may determine the radius information in various ways, such as calculating the average or the median of the calculated distances.

The electronic device may change the radius information of the nodes at the boundary of the open vessel to a preset value. For example, the preset value may be ‘0’. Accordingly, in the completed network, the electronic device may use the preset value to select the boundary nodes among the nodes.

The electronic device may calculate the length of the edge. For example, a first edge may be a line connecting the first node and the second node. The electronic device may determine the length of the first edge based on the coordinates of the first node and the second node.

In this manner, the electronic device may generate the vessel network 500 by generating nodes and edges, and may determine the coordinates of the nodes, the radius information of the nodes, the length of the edges, etc.

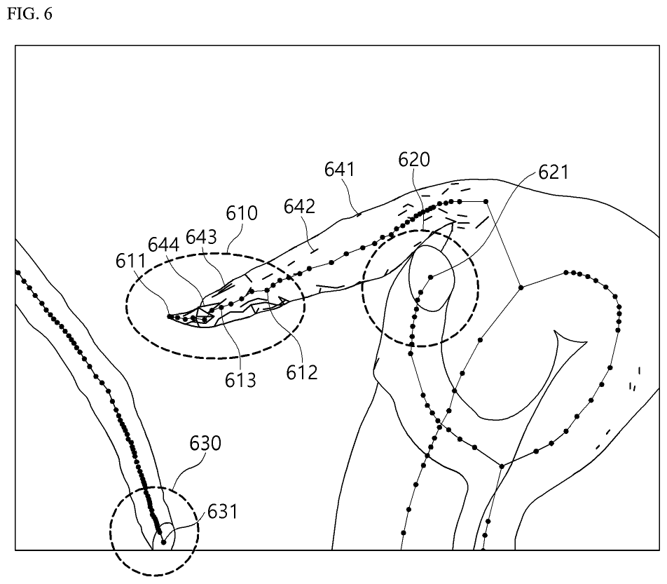

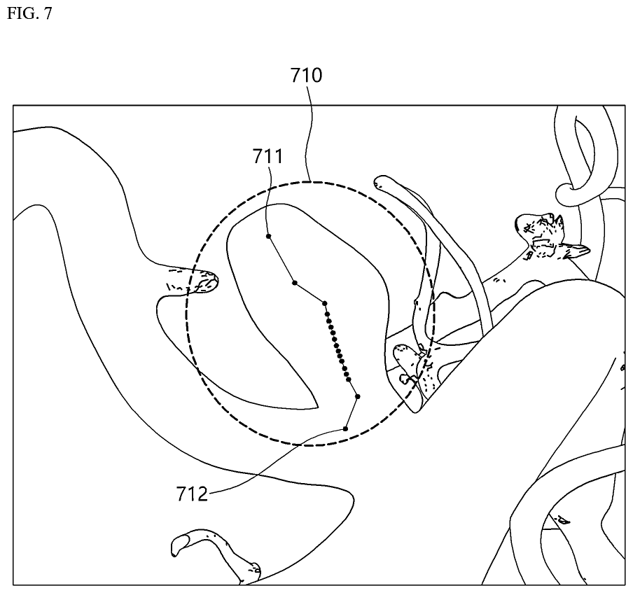

The electronic device may determine a leaf node in the vessel network 500 (S123). Referring to FIG. 6, the configuration for determining the leaf node 611 according to one embodiment can be confirmed. The electronic device may determine a candidate leaf vessel from among the vessels at the ends of the vessel mesh that are not open vessels 620, 630. The candidate leaf vessel may have only one neighboring node. The electronic device may determine, as a candidate leaf node, a node among the plurality of nodes that has only one neighboring node and does not correspond to an open vessel 620, 630.

The electronic device may determine a leaf vessel 610 from among the candidate leaf vessels and then determine a leaf node 611 within the leaf vessel 610. In the vessel mesh, vessels at the ends that are not open vessels 620, 630 may include a leaf vessel 610, an aneurysm, and the like. That is, the leaf vessel 610, the aneurysm, and the like can be the candidate leaf vessels. The leaf vessel 610 may refer to a vessel whose diameter gradually decreases and converges to a single point.

The electronic device may filter the boundary nodes 621 and 631 in the open vessels 620, 630 using a preset value (for example, the aforementioned ‘0’). This is because the electronic device has determined the radius information of the boundary nodes 621 and 631 to be the preset value. On the other hand, the leaf node 611 in the leaf vessel 610 may not be filtered out because its radius information has a value other than ‘0’. Accordingly, the electronic device may determine the leaf node 611 to filter the leaf vessel 610.

As the nodes in the leaf vessel 610 get closer to the leaf node 611 (for example, from node 612 to node 613, and from node 613 to node 611), the angle between the cells around the nodes may become smaller. The angle may also be referred to as a dihedral angle. The electronic device may calculate the angle (or dihedral angle) between two adjacent cells on the surface and detect two cells where the angle is equal to or less than (or is less than) a reference angle. The electronic device may display the tangents 641 to 644 of the two detected cells. In FIG. 6, for convenience of explanation, only four tangents 641 to 644 are labeled with reference numbers, but many other tangents that are not labeled with reference numbers are also shown. In FIG. 6, it can be seen that the leaf vessel 610 includes a relatively large number of tangents 641 to 644, while the open vessels 620, 630 include relatively fewer tangents.

The electronic device may calculate the distance between the tangents and the nodes. For example, the electronic device may calculate the distances between the first node 612 and the n tangents (where n is an integer greater than 1) around the first node 612. The electronic device may calculate the distances between the second node 613 and the n tangents around the second node 613. The electronic device may calculate the distances between the leaf node 611 and the n tangents around the leaf node 611.

The electronic device may determine the shortest distance from the node to the tangent as the distance between the node and the tangent. However, the embodiments are not limited thereto, various points within the tangent, such as the midpoint of the tangent, may also be used for distance calculation.

The electronic device may determine the leaf node 611 among the nodes based on the calculated distances. In some embodiments, the electronic device may determine a node as the leaf node 611 if the calculated distance is equal to or less than (or is less than) a reference value. In some embodiments, the electronic device may determine the node with the shortest calculated distance as the leaf node 611.

The electronic device may also determine the leaf node 611 using the Frobenius norm as shown in Equation 1.

$${norm}_{f} = \sqrt{{d}_{1}^{2} + {d}_{2}^{2} + … + {d}_{n}^{2}} , \qquad [ Equation 1 ]$$

$$where {d}_{i}^{2} = ‖ leaf node coord - point i coord ‖$$

Here, normf may indicate the Frobenius norm value of an arbitrary node f among the nodes of the vessel network, and is calculated based on the distances between the node f and each of the n tangents. d_i\hat{ }2 may indicate the norm of a vector defined by the coordinates of the candidate leaf node (referred to as “leaf node coord”) and the coordinates of a point i on the tangent (referred to as “point i coord”), where the point i may indicate a point on the tangent that has the shortest distance to the candidate leaf node.

The electronic device may determine the Frobenius norm values of the nodes and may determine a node having a Frobenius norm value that is equal to or less than (or is less than) a reference value as a leaf node 611. The electronic device may change the radius information of the leaf node 611 to a preset value (for example, ‘0’). Accordingly, the electronic device may filter the leaf node 611 and the boundary nodes 621, 631 from the vessel network using the preset value.

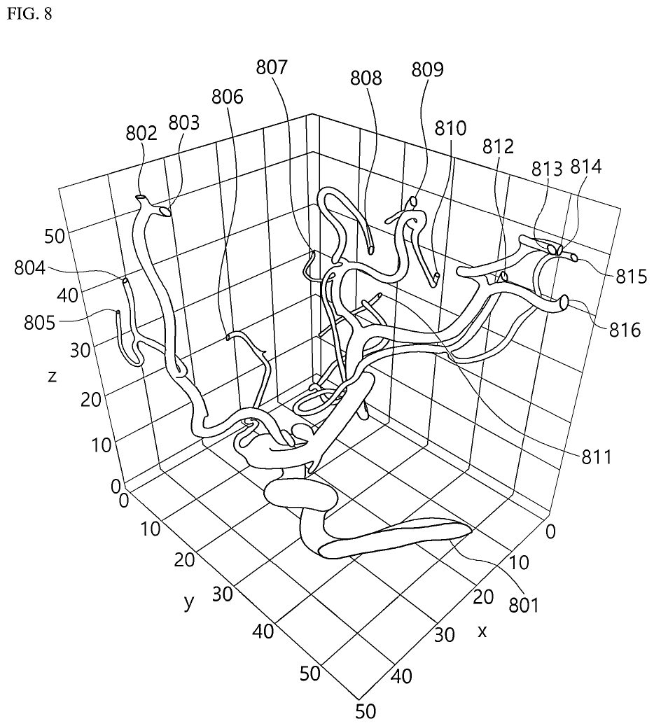

Referring again to FIG. 3, the electronic device may determine the aneurysm network (S124). Referring to FIG. 7, the configuration for determining the aneurysm network according to an embodiment can be confirmed. The electronic device may determine a blood vessel at the ends of the vessel mesh, which is neither a leaf vessel 610 nor an open vessel 620, 630, as an aneurysm candidate 710.

The electronic device may verify the aneurysm candidate 710, and if the verification is successful, may determine the aneurysm candidate 710 as an aneurysm. For example, the electronic device may detect a start node 711 at the end of the aneurysm candidate 710. The electronic device may start from the start node 711 and reach a branch node 712. The branch node 712 may refer to a node with three or more neighboring nodes. In FIG. 7, although other nodes are not illustrated to explain the path from the start node 711 to the branch node 712, in practice, the branch node 712 may be connected to three or more nodes.

The electronic device may determine that the verification of the aneurysm candidate 710 is successful if the aneurysm candidate 710 includes the branch node 712. The inclusion of the branch node 712 within the aneurysm candidate 710 may mean that the branch node 712 exists within a reference distance from the start node 711. The reference distance may refer to the lengths of the edges between the start node 711 and the branch node 712, but in some embodiments, the reference distance may also refer to the straight-line distance between the start node 711 and the branch node 712. The electronic device may determine that the verification of the aneurysm candidate 710 has failed if the aneurysm candidate 710 does not include the branch node 712.

The electronic device may generate the aneurysm network that includes the nodes and edges extending from the start node 711 to the branch node 712 of the aneurysm.

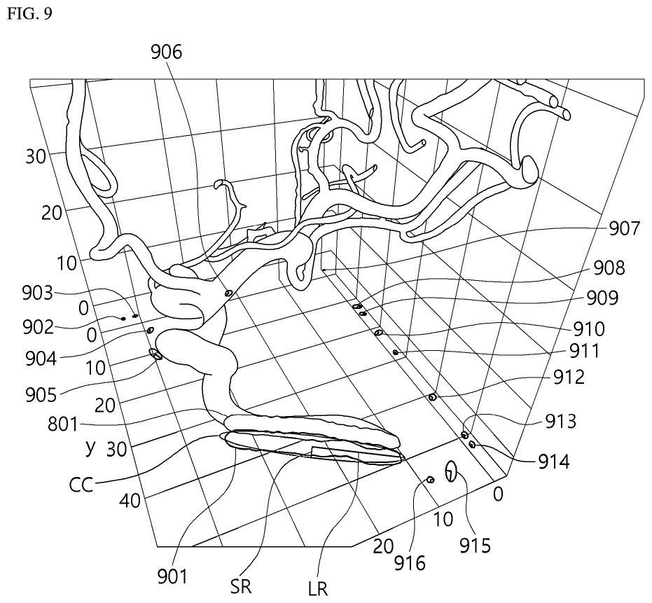

Referring again to FIG. 3, the electronic device may determine the flow network (S125). The flow network may represent the flow of blood within the blood vessels. In FIGS. 8 and 9, the configuration for determining the flow network according to an embodiment can be confirmed.

Referring to FIG. 8, the electronic device may detect the open vessels 801 to 816 in the vessel network. The open vessels 801 to 816 may refer to the blood vessels at the ends of the vessel network. The blood vessels at the ends of the vessel network may further include a leaf vessel, an aneurysm, and others. The electronic device may filter out the leaf vessel, the aneurysm, and others from the end vessels of the vessel network using the method described with reference to FIGS. 6 and 7, and extract the open vessels 801 to 816. The electronic device may determine a node of the largest blood vessel among the open vessels 801 to 816 as the start node.

In some embodiments, the electronic device may project the open vessels 801 to 816 onto an ellipse model. Projecting the open vessels 801 to 816 may also be understood as finding an ellipse model that best fits the open vessels 801 to 816. For example, the electronic device may project the open vessels 801 to 816 onto an ellipse model using an open-source library of image processing. The electronic device may obtain the parameters of each ellipse. The parameters of the ellipse may include values such as the semi-minor axis (or minor axis), the semi-major axis (or major axis), the circumference, and the like.

Referring to FIGS. 8 and 9, the electronic device may determine ellipse models 901 to 916 onto which the open vessels 801 to 816 are projected. The electronic device may obtain the parameters of the ellipse models 901 to 916. For example, in case of the ellipse model 901, it may have parameters such as the semi-minor axis (SR), the semi-major axis (LR), and the circumference (CC).

In some embodiments, the electronic device may obtain the semi-minor axis of the ellipse models 901 to 916 and may select the ellipse model 901 with the largest semi-minor axis among them. The electronic device may determine the open vessel 801 corresponding to the ellipse model 901 as a start vessel. The electronic device may determine the node of the start vessel as the start node.

The electronic device may perform node propagation starting from the start node. The electronic device may determine the direction to subsequent nodes as the flow of blood. The electronic device may display the flow of blood. For example, if the electronic device propagates from the first node to the second node, an arrow may be displayed from the first node to the second node. That is, an edge between the first node and the second node may be displayed as an arrow.

The electronic device may reach the branch node while performing node propagation. For example, the branch node may be connected to a third node, a fourth node, and a fifth node, where the third node is the node prior to the branch node, and the fourth and fifth nodes are nodes after the branch node. The electronic device may determine which node to perform node propagation on first between the fourth node and the fifth node. In some embodiments, the electronic device may perform node propagation first on the node with the larger radius between the fourth node and the fifth node. For example, if the fourth node has a larger radius than the fifth node, the electronic device may first perform node propagation from the branch node towards the fourth node, complete the blood flow corresponding to the fourth node, and then perform node propagation from the branch node towards the fifth node, and complete the blood flow corresponding to the fifth node. However, the embodiments are not limited thereto, and the electronic device may also first perform node propagation towards the node with the smaller radius from the branch node.

The electronic device may generate a flow network that includes the flow of blood within the vessel network.

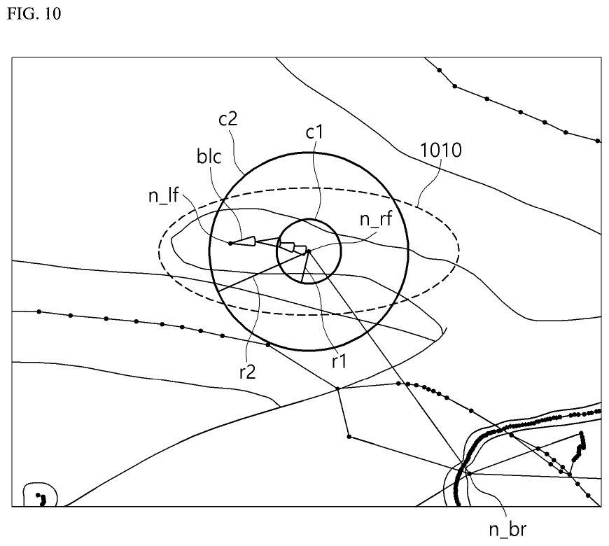

Referring again to FIG. 3, the electronic device may remove noise 1010 from the flow network (S126). Referring to FIG. 10, the configuration for determining noise according to an embodiment can be confirmed. First, the electronic device may filter out at least one of open vessels, leaf vessels, and aneurysms from the blood vessels at the ends of the vessel network. In other words, blood vessels at the ends that are not open vessels, leaf vessels, or aneurysms may be candidates for noise.

The electronic device may determine a leaf node (n_lf) of a candidate noise. The electronic device may perform node propagation in the reverse direction of the blood flow (blc), starting from the leaf node (n_lf). The electronic device may continue node propagation until it reaches the branch node (n_br). Among the paths from the leaf node (n_lf) to the branch node (n_br), the electronic device may determine the node just before the branch node (n_br) as a reference node (n_rf).

The electronic device may determine whether the candidate noise is actual noise based on the reference node (n_rf). For example, the electronic device may generate a sphere (c1) based on a radius (r1) of the reference node (n_rf). The electronic device may obtain a radius (r2) by multiplying the radius (r1) by a predetermined weight, and generate a sphere (c2) based on the radius (r2). If the leaf node (n_lf) is located inside the sphere (c2), the electronic device may determine that the candidate noise as actual noise. If the leaf node (n_lf) is located outside the sphere (c2), the electronic device may determine that the candidate noise is not actual noise.

The electronic device may determine the path from the leaf node (n_lf) to the reference node (n_rf) as a noise 1010. The electronic device may also classify the noise 1010 as a sticky network. The electronic device may remove the noise 1010 including the nodes from the leaf node (n_lf) to the reference node (n_rf), from the flow network.

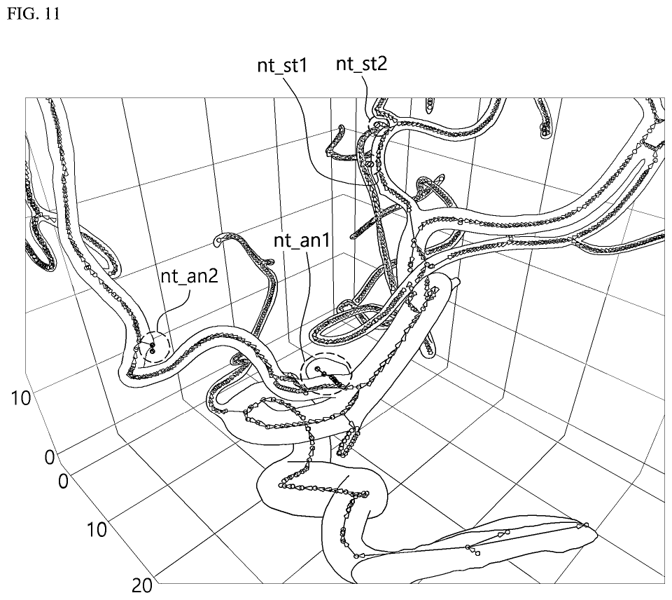

Referring to FIG. 11, it can be confirmed that aneurysm networks (nt_an1, nt_an2) and sticky networks (nt_st1, nt_st2) are displayed in a flow network according to an embodiment. The description of step (S124) in FIG. 3 may apply to the aneurysm networks (nt_an1, nt_an2), and the description of step (S126) in FIG. 3 may apply to the sticky networks (nt_st1, nt_st2). The electronic device may filter the aneurysm networks (nt_an1, nt_an2) and the sticky networks (nt_st1, nt_st2) from the flow network.

In other words, the electronic device may obtain third nodes by excluding the first nodes of the aneurysm networks (nt_an1, nt_an2) and the second nodes of the sticky networks (nt_st1, nt_st2) from the plurality of nodes included in the flow network. The electronic device may generate a flow network based on the third nodes. That is, the electronic device may not determine blood flow in the first nodes and the second nodes.

The electronic device may generate the flow network and perform step (S130) of FIG. 2.

In FIG. 3, the steps (S121 to S126) are illustrated as being included in step (S120) of FIG. 2, however, the embodiments are not limited thereto, and step (S120) may be implemented to include any combination of the steps (S121 to S126). For example, step (S120) may be implemented to include steps (S121 to S125).

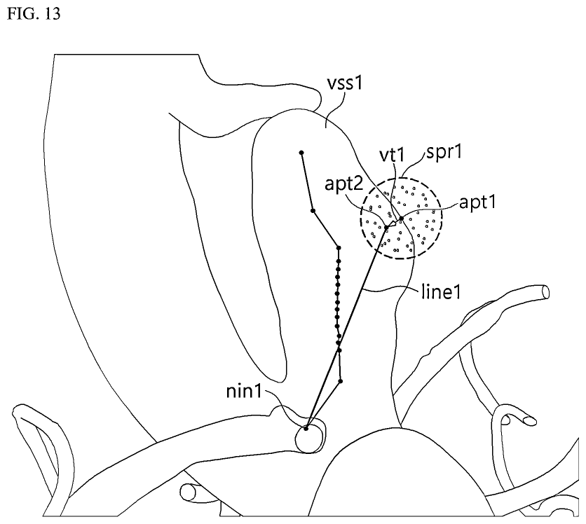

FIG. 12 is an example flowchart showing a method for processing a vessel network by an electronic device according to an embodiment, and FIGS. 13 to 18 illustrate a method for processing a vessel network by an electronic device according to an embodiment.

Referring to FIG. 12, an electronic device according to an embodiment may generate a vessel network (S120) and may determine a base node (S131).

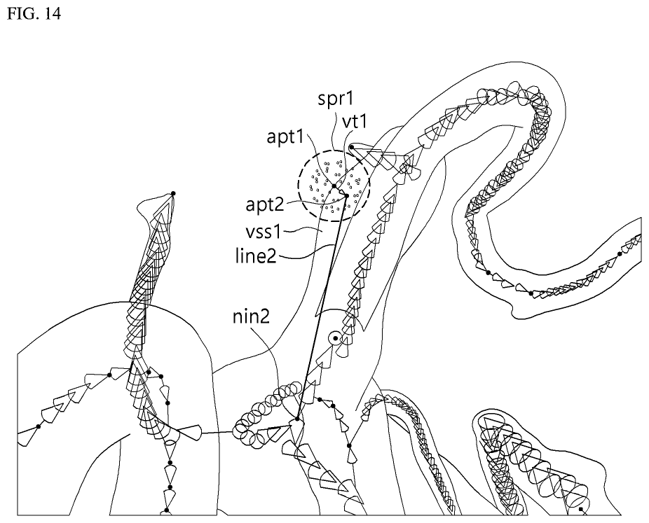

FIGS. 13 and 14 illustrate the configuration for determining a base node according to an embodiment. Referring to FIG. 13, an electronic device according to an embodiment may determine an aneurysm point (apt1). The aneurysm point (apt1) may be located on a vessel surface (vss1). In some embodiments, the electronic device may receive user input and may determine the aneurysm point (apt1) based on the user input. For example, the electronic device may display a vessel image, and the user may recognize an aneurysm in the vessel image and select the aneurysm point (apt1) corresponding to the aneurysm. The vessel image may refer to the vessel mesh, the vessel network, or other images described above.

In some embodiments, the electronic device may determine the aneurysm point (apt1) using Artificial Intelligence (AI). The electronic device may include a trained artificial neural network configured to output an aneurysm point (for example, apt1) from a vessel image. The trained artificial neural network may have parameters (or weights) for outputting the aneurysm point.

The electronic device may generate a spherical point cloud (spr1) based on the aneurysm point (apt1). The electronic device may generate the point cloud (spr1) centered on the aneurysm point (apt1) with a predetermined radius. The point cloud (spr1) may include first points located inside a vessel surface (vss1) and second points located outside the vessel surface (vss1).

The electronic device may generate a vector (vt1) based on the first points of the point cloud (spr1). For example, the electronic device may calculate vectors for the first points of the aneurysm point (apt1) and determine the average of the calculated vectors as the vector (vt1). The electronic device may move the aneurysm point (apt1) to a new point (apt2) based on the vector (vt1).

The electronic device may generate line segments connecting each node of the aneurysm network and the point (apt2). The description provided for the aneurysm network with reference to FIG. 7 may apply here as well. For example, the electronic device may generate a line (line1) based on a node (nin1) and the point (apt2). The electronic device may determine whether the line (line1) passes through the vessel surface (vss1). If the line (line1) does not pass through the vessel surface (vss1), the electronic device may determine a start node (for example, the node (nin1)) of the aneurysm network as a first candidate base node. The start node may refer to a node of the aneurysm network directly connected to a branch node of a flow network. If the line (line1) passes through the vessel surface (vss1), the electronic device may determine whether the lines generated based on other nodes of the aneurysm network pass through the vessel surface (vss1). At this time, if at least one line does not pass through the vessel surface (vss1), the electronic device may determine the start node of the aneurysm network as the first candidate base node.

FIG. 14 is a diagram obtained by rotating FIG. 13 to change a viewing point. Referring to FIG. 14, the electronic device may determine a branch node (nin2) in the flow network that is connected to the aneurysm network. The electronic device may generate a line (line2) based on the branch node (nin2) and the point (apt2). The electronic device may determine whether the line (line2) passes through the vessel surface (vss1). If the line (line2) does not pass through the vessel surface (vss1), the electronic device may determine the branch node (nin2) as a second candidate base node. If the line (line2) passes through the vessel surface (vss1), the electronic device may not determine the branch node (nin2) as the second candidate base node.

The electronic device may determine, as a base node, a node with a shorter line (line1 or line2) between the first candidate base node (for example, the start node of the aneurysm network) from FIG. 13 and the second candidate base node (for example, the branch node of the flow network) from FIG. 14.

Referring back to FIG. 12, the electronic device may generate a maxcut network (S132).

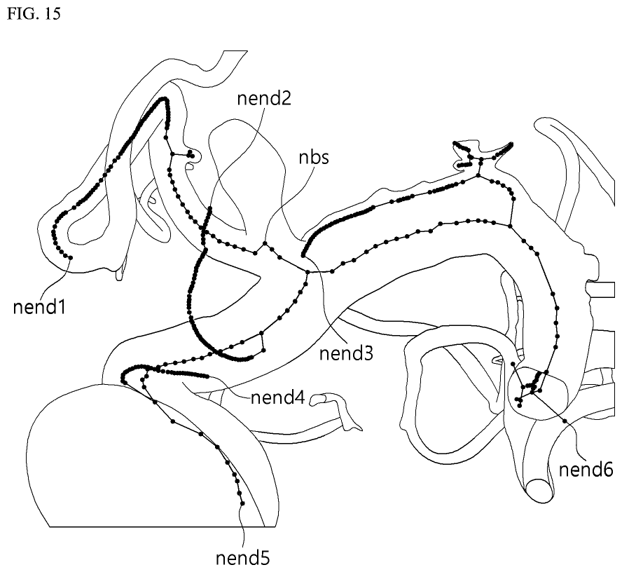

FIG. 15 illustrates the configuration for generating the maxcut network according to an embodiment. Referring to FIG. 15, the electronic device may generate the maxcut network from the flow network based on a base node (nbs). The maxcut network is a network comprising an aneurysm network within the flow network, and the electronic device may generate the maxcut network by performing pruning on the flow network.

The electronic device may determine nodes (nend1 to nend6) that are located at a predetermined distance from the base node (nbs). Specifically, the distance from each of the nodes (nend1 to nend6) to the base node (nbs) may be substantially the same. The distance may be calculated as the sum of the edges between nodes. The electronic device may determine the nodes from the base node (nbs) to the nodes (nend1 to nend6) as the maxcut network.

Since it would be inefficient in terms of time and cost for the electronic device to examine all the nodes in the flow network due to the vast amount of computation required, the electronic device may generate the maxcut network and verify the nodes within the maxcut network to generate the aneurysm region.

In FIG. 15, for the convenience of explanation, only nodes nend1 to nend6 are shown as nodes located at a predetermined distance from the base node (nbs), but there may be at least one additional node.

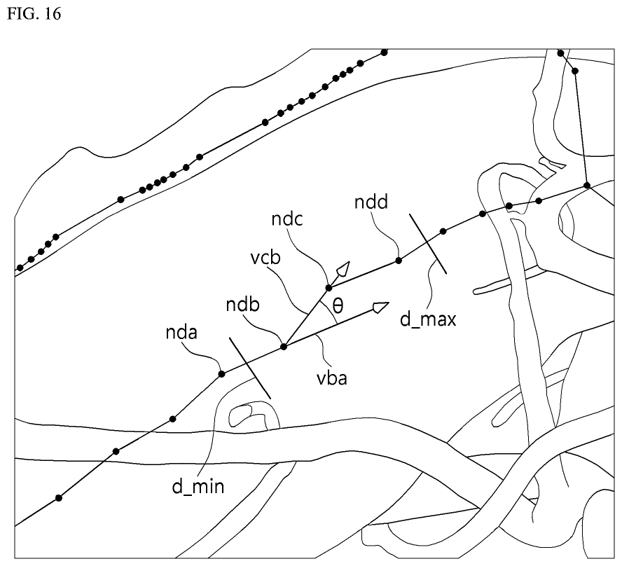

Referring back to FIG. 12, the electronic device may determine a clip node (S133). Referring to FIG. 16 together, the configuration for determining the clip node can be confirmed. The electronic device may determine the clip node from among nodes (ndb, ndc, ndd) located within a predetermined distance range from the base node. The predetermined distance range may include a minimum distance (d_min) and a maximum distance (d_max). That is, the electronic device may verify the nodes (ndb, ndc, ndd) that are at a distance from the base node within the minimum distance (d_min) to the maximum distance (d_max).

The electronic device may begin the verification starting with the node (ndb). The electronic device may generate vectors (vba, vcb) based on the node (ndb). For example, the electronic device may generate the vector (vba) based on the nodes (nda, ndb) and generate the vector (vcb) based on the nodes (ndb, ndc). The electronic device may calculate the angle (θ) between the vectors (vba, vcb).

The electronic device may determine whether the angle (θ) exceeds a first angle. If the angle (θ) does not exceed the first angle, the electronic device may determine the node (ndb) as the clip node.

If the angle (θ) exceeds the first angle, the electronic device may adjust the minimum distance (d_min). The electronic device may increase the minimum distance (d_min). For example, the electronic device may adjust the minimum distance (d_min) as shown in Equation 2. The electronic device may calculate the angle (θ) while performing node propagation (i.e., exploring nodes), and if the angle (θ) meets a predetermined condition (for example, the angle (θ) exceeds the first angle), the electronic device may adjust the minimum distance (d_min).

$${min}_{n d b} = {min}_{n d a} + r * interp ( θ , [ {θ}_{a} , {θ}_{b} ] , [ α , β ] ) \qquad [ Equation 2 ]$$

Herein, min_{ndb} may indicate the minimum distance at node (ndb), and min_{nda} may indicate the minimum distance at node (nda) and correspond to the minimum distance (d_min). Node (ndb) is a node that comes after node (nda), and r may indicate radius information of node (ndb). The term “interp(θ,[θₐ,θ_{b}], [α,β])” is an interpolation function that outputs a value between α and β depending on the value of the angle θ. Here, β may be greater than α and θ_{b} may be greater than θₐ. For example, if the angle θ is θₐ, the interpolation function outputs α; if the angle θ is θ_{b}, the interpolation function outputs β; and if the angle θ is between θₐ and θ_{b}, the interpolation function outputs a value between α and β. In some embodiments, the interpolation function could be a first-order linear function with a slope of (β−α)/(θ_{b}−θₐ). Depending on the embodiment, the interpolation function can be implemented in various ways.

The electronic device may perform the verification of node (ndc) based on the adjusted minimum distance. Similar to the process described for node (ndb), the electronic device may generate a first vector based on nodes (ndc, ndd) and calculate the angle between the first vector and vector (vcb). The electronic device may determine node (ndc) as the clip node based on the calculated angle.

If the electronic device does not determine node (ndc) as the clip node, it may determine node (ndd), which is the node just before the maximum distance (d_max), as the clip node.

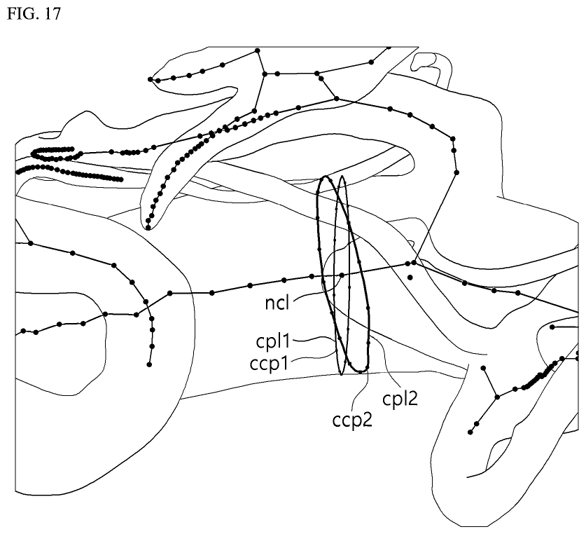

Referring back to FIG. 12, the electronic device may determine a cutting plane (S134). Referring to FIG. 17 together, the configuration for determining the cutting plane according to an embodiment can be confirmed. The electronic device may generate cutting plane candidates (cpl1, cpl2) based on a clip node (ncl). For example, the electronic device may generate a first vector based on the clip node (ncl) and a node immediately preceding the clip node, and generate a second vector based on the clip node (ncl) and a node immediately following the clip node. The electronic device may then generate a first cutting plane candidate (cpl1), which is a plane having the first vector as its normal vector, and generate a second cutting plane candidate (cpl2), which is a plane having the second vector as its normal vector.

The electronic device may display contact points where the cutting plane candidates (cpl1, cpl2) intersect with the surface. For example, the electronic device may display multiple contact points (ccp1) where the first cutting plane candidate (cpl1) contacts the surface, and multiple contact points (ccp2) where the second cutting plane candidate (cpl2) contacts the surface.

The electronic device may determine the number of cutting plane candidates to generate based on the angle between the first vector and the second vector. For example, if the angle is large, the electronic device may generate a relatively large number of cutting plane candidates, whereas if the angle is small, the electronic device may generate relatively fewer cutting plane candidates.

The electronic device may determine the number of cutting plane candidates based on Equation 3.

$$n\_plane = interp ( θ , [ {θ}_{c} , {θ}_{d} ] , [ A , B ] ) \qquad [ Equation 3 ]$$

Herein, n_plane may indicate the number of cutting plane candidates that the electronic device will generate at the clip node (ncl). The term “interp(θ,[θ_{c},θ_{d}],[A, B])” is an interpolation function that outputs a value between A and B depending on the value of the angle θ. The angle θ is the angle between the first vector and the second vector, as previously explained with reference to FIG. 16. Here, B may be greater than A. In some embodiments, A may be an integer greater than or equal to 2. The value θ_{d} may be greater than θ_{c}. For example, if the angle θ is θ_{c}, the interpolation function outputs A; if the angle θ is θ_{d}, the interpolation function outputs B; and if the angle θ is between θ_{c} and θ_{d}, the interpolation function outputs a value between A and B. In some embodiments, the interpolation function could be a first-order linear function with a slope of (B−A)/(θ_{d}−θ_{c}). Depending on the embodiment, the interpolation function can be implemented in various ways.

The electronic device may further generate at least one plane between the first cutting plane candidate (cpl1) and the second cutting plane candidate (cpl2) based on the number of cutting plane candidates. For example, the electronic device may further generate a third vector between the first vector and the second vector and further generate a plane using this third vector as its normal vector. The electronic device may use an interpolation function of a first-order linear function to generate the third vector.

The electronic device may determine a cutting plane from among the cutting plane candidates (cpl1, cpl2). The electronic device may project each of the cutting plane candidates (cpl1, cpl2) onto elliptical models. The electronic device may obtain the semi-major axis and semi-minor axis from each projected elliptical model. In some embodiments, the electronic device may determine a cutting plane candidate as a final cutting plane if the ratio of the semi-minor axis to the semi-major axis exceeds a predetermined value.

For example, the electronic device may obtain the first semi-major axis and the first semi-minor axis corresponding to the first cutting plane candidate (cpl1) and the second semi-major axis and the second semi-minor axis corresponding to the second cutting plane candidate (cpl2). The electronic device may calculate the ratio of the first semi-minor axis to the first semi-major axis (i.e., ‘the first semi-minor axis/the first semi-major axis’) and the ratio of the second semi-minor axis to the second semi-major axis (i.e., ‘the second semi-minor axis/the second semi-major axis’). The electronic device may determine the cutting plane based on the calculated values.

In some embodiments, the electronic device may determine, as a cutting plane, a cutting plane candidate with the largest ratio of ‘semi-minor axis/semi-major axis’.

The electronic device may re-determine a cutting plane if there is an edge passing through the determined cutting plane. For example, a retrograde edge may pass through the cutting plane. In some embodiments, the electronic device may re-determine a clip node.

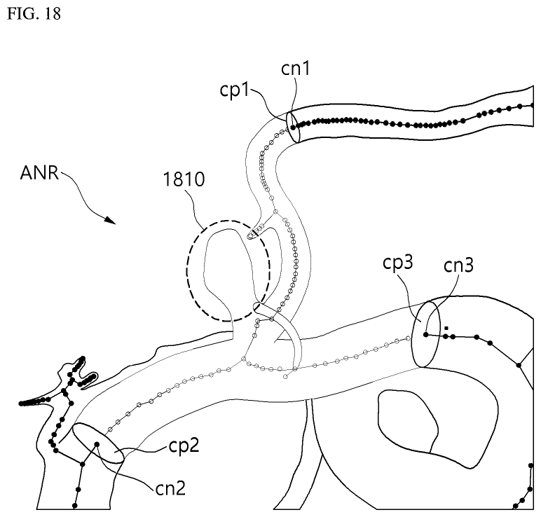

Referring back to FIG. 12, the electronic device may generate an aneurysm region (S135). Referring to FIG. 18 together, the configuration for generating the aneurysm region can be confirmed. The electronic device may cut the vessel network (or flow network) by using clip nodes cn1 to cn3 and cutting planes (cp1 to cp3) determined based on the clip nodes cn1 to cn3. As a result, the electronic device can generate an aneurysm region (ANR) including an aneurysm network 1810.

The electronic device may display the generated aneurysm region (ANR). For example, the electronic device may highlight the aneurysm region (ANR) within the vessel network by displaying it with different colors, textures, or other visual distinctions. This allows the user to more easily analyze the aneurysm region (ANR), which is the area of interest. As the electronic device precisely isolates the aneurysm region (ANR), it enables the user to accurately measure parameters of the aneurysm region (ANR). Consequently, the user can make a precise diagnosis and develop an appropriate treatment plan based on the measured parameters.

In FIG. 12, the steps (S131 to S135) are illustrated as being included in step (S130) of FIG. 2, however, the embodiments are not limited thereto, and step (S130) may be implemented to include any combination of the steps (S131 to S135). For example, step (S130) may be implemented to include steps (S131 and S133 to S135).

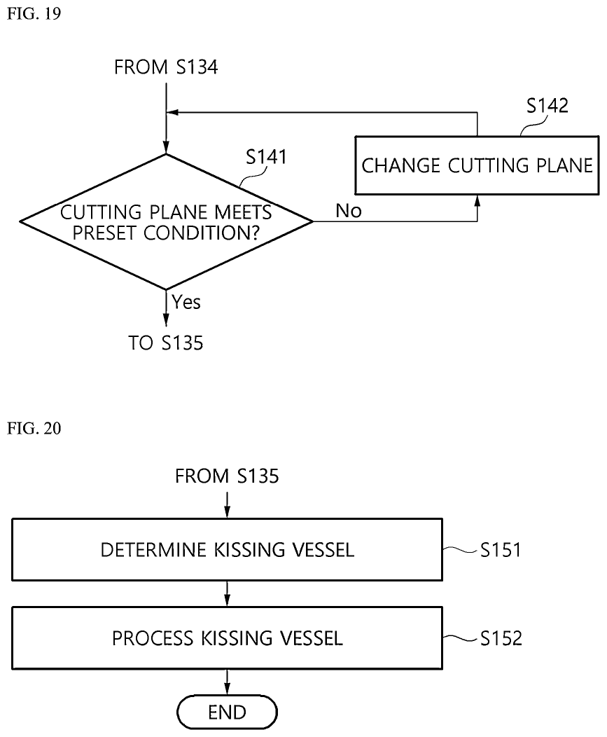

FIG. 19 is an example flowchart showing a method for processing a vessel network by an electronic device according to an embodiment.

Referring to FIG. 19, the electronic device according to an embodiment may determine a cutting plane (S134) and determine whether the cutting plane meets a predetermined condition (S141). That is, the electronic device may verify the determined cutting plane.

In some embodiments, the electronic device may determine whether the clip node of the determined cutting plane is the branch node. The electronic device may determine the cutting plane does not meet the predetermined condition if the clip node is the branch node. The electronic device may determine the cutting plane meets the predetermined condition if the clip node is not the branch node.

In some embodiments, the electronic device may determine a midpoint (center point) of the determined cutting plane. The electronic device may determine the distance between the midpoint and the clip node. The electronic device may determine the cutting plane meets the predetermined condition if the distance is less than or equal to (or is less than) a predetermined value. The electronic device may determine the cutting plane does not meet the predetermined condition if the distance is greater than (or is greater than or equal to) the predetermined value.

In some embodiments, the electronic device may determine whether the semi-major axis, the semi-minor axis, and the radius of the clip node of the determined cutting plane meet a predetermined condition. The electronic device may determine the cutting plane meets the predetermined condition if the sum of a difference between the semi-major axis and the radius (a first difference) and a difference between the semi-minor axis and the radius (a second difference) is less than or equal to (or is less than) a predetermined value. The electronic device may determine the cutting plane does not meet the predetermined condition if the sum of the first and second differences is greater than (or is greater than or equal to) the predetermined value. In this embodiment, although the configuration in which the electronic device determines the satisfaction of the predetermined condition based on the sum of the first and second differences is described, the embodiment is not limited thereto, and the electronic device may perform the determination in various manners, such as by performing the determination based on the sum of the squares of the first and second differences.

In the present invention, the predetermined condition is described using the branch node, the midpoint, the semi-major axis, the semi-minor axis, and the radius, but the predetermined condition may be implemented as various combinations of the aforementioned conditions or may further include at least one other condition. For example, the electronic device may perform verification based on the midpoint, the semi-major axis, the semi-minor axis, and the radius.

If the cutting plane meets the predetermined condition, the electronic device may generate an aneurysm region based on the cutting plane (S135). For example, the electronic device may generate an aneurysm region (ANR) as shown in FIG. 18.

If the cutting plane does not meet the predetermined condition, the electronic device may modify (or change) the cutting plane (S142). The electronic device may determine cutting planes for each node between the clip node and the base node. The electronic device may calculate costs of the nodes and determine a node with the lowest cost as a new clip node.

In some embodiments, the electronic device may perform verification of the nodes based on Equation 4.

$$cost = {( a - r )}^{2} + {( b - r )}^{2} + {d}_{nc}$$

$${d}_{nc} = ‖ node point - ellipse center point ‖$$

Herein, cost may indicate score of the node to be verified, a is the semi-major axis of the elliptical model of the cutting plane of the node to be verified, b is the semi-minor axis of the elliptical model, r is the radius of the clip node of the cutting plane, and d_{nc} may be the distance between the node to be verified and the ellipse center point (or the midpoint of the elliptical model).

The electronic device may determine the node with the lowest cost among the nodes as a new clip node. The electronic device may determine whether the cutting plane of the new clip node meets the predetermined condition. In some embodiments, the electronic device may skip the verification of the cutting plane of the new clip node. In this case, the electronic device may generate an aneurysm region based on the cutting plane of the new clip node.

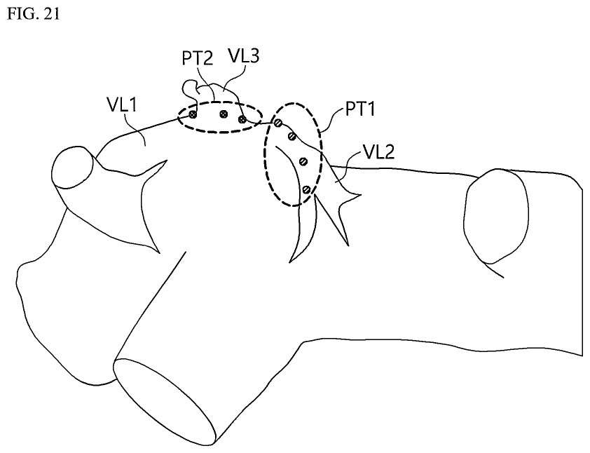

FIG. 20 is an example flowchart showing a method for processing a kissing vessel by an electronic device according to an embodiment, and FIGS. 21 to 26 illustrate a method for processing a kissing vessel by an electronic device according to an embodiment.

Referring to FIGS. 20 and 21, the electronic device according to an embodiment may generate the aneurysm region (S135) and determine kissing vessels (VL1 to VL3) (S151).

The kissing vessels (VL1 to VL3) may refer to blood vessels that are actually separated from other blood vessels within the human body but appear to be attached due to artifacts, noise, or other reasons during image processing procedures. If the kissing vessels (VL1 to VL3) is present near the aneurysm, it may cause errors in the quantitative analysis of the aneurysm, leading to an image inaccurate and unintended. A first kissing vessel (VL1) among the kissing vessels (VL1 to VL3) may be the aneurysm.

In some embodiments, the electronic device may determine a kissing vessel based on user input. The user may transmit the user input corresponding to the kissing vessel to the electronic device, and the electronic device may receive the user input.

In some embodiments, the electronic device may determine the kissing vessel using artificial intelligence. The electronic device may include an artificial neural network trained to output the kissing vessel within the vessel network. The trained artificial neural network may have parameters for outputting the kissing vessel.

The electronic device may determine the points where the kissing vessels (VL1 to VL3) makes contact. For example, the electronic device may determine points (PT1) where the first kissing vessel (VL1) and a second kissing vessel (VL2) make contact, and determine points (PT2) where the first kissing vessel (VL1) and the third kissing vessel (VL3) make contact. Depending on the embodiment, the points (PT1, PT2) may also be determined based on user input.

In some embodiments, step (S151) may be performed after step (S120) of FIG. 2. In this way, if the electronic device has generated the vessel network, the timing of performing step (S151) is not particularly restricted. The electronic device may process the kissing vessels (VL1 to VL3)

(S152). Processing the kissing vessels (VL1 to VL3) can be understood as an operation to separate the attached kissing vessels (VL1 to VL3). The electronic device may determine a cutting line to separate the kissing vessels (VL1 to VL3).

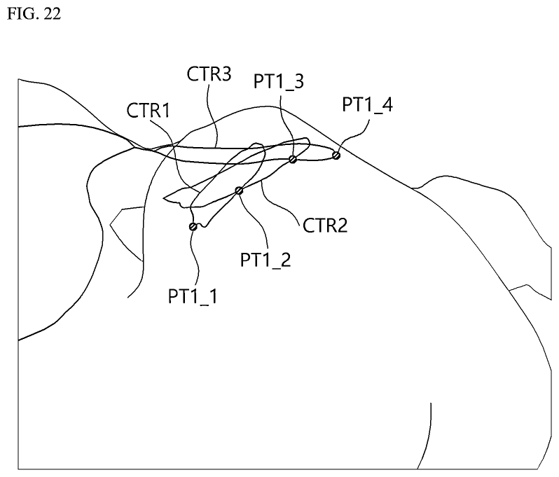

The electronic device may determine the cutting line based on the points (PT1, PT2). For example, the electronic device may determine a first cutting line based on the first points (PT1) and determine a second cutting line based on second points (PT2). The configuration in which the electronic device determines the first cutting line based on the first points PT1 will be described with reference to FIG. 22.

Referring to FIG. 22, the electronic device may generate contours (CTR1 to CTR3) based on the first points (PT1) and a camera position point. The first points (PT1) may include a plurality of points (PT1_1 to PT1_4).

camera position points may refer to positions of the camera corresponding to each of the points (PT1_1 to PT1_4). For example, when the user inputs the points (PT1_1 to PT1_4) into the electronic device, the user may input the points (PT1_1 to PT1_4) while rotating the vessel network. At this time, the point from which the user views the vessel network while inputting the points (PT1_1 to PT1_4) may be the camera position points. There may be a first camera position point corresponding to the point (PT1_1), a second camera position point corresponding to the point (PT1_2), a third camera position point corresponding to the point (PT1_3), and a fourth camera position point corresponding to the point (PT1_4).

The electronic device may generate a plane using three points and generate a contour by forming a line where the generated plane intersects the surface of the vessel network. For example, the electronic device may generate a first contour (CTR1) using the points (PT1_1 and PT1_2) and the second camera position point. Depending on the embodiment, the first camera position point may be used instead of the second camera position point.

In the same manner, the electronic device may generate a second contour (CTR2) using the points (PT1_2 and PT1_3) and the third camera position point. The electronic device may generate a third contour (CTR3) using the points (PT1_3 and PT1_4) and the fourth camera position point.

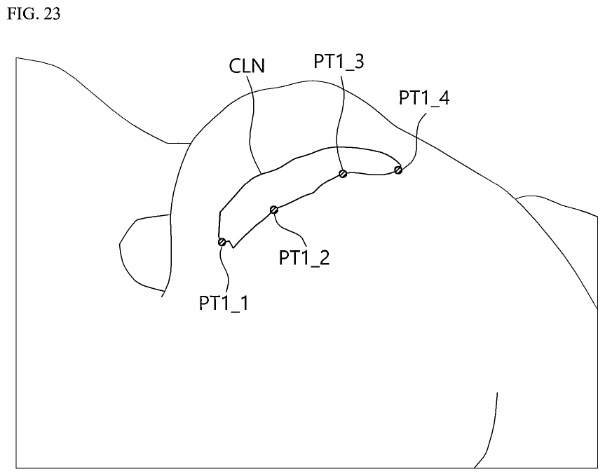

The electronic device may determine the first cutting line based on the contours (CTR1 to CTR3). The electronic device may remove the line components extending outward from the aneurysm in the contours (CTR1 to CTR3). The electronic device may use normal vectors of the contours (CTR1 to CTR3). The electronic device may generate the first cutting line by connecting the remaining lines after removing the line components from the contours (CTR1 to CTR3). The first cutting line generated by the electronic device may be as shown in FIG. 23.

Referring to FIG. 23, the electronic device may generate a first cutting line (CLN). The electronic device may perform image processing on the surface of the vessel network based on the first cutting line (CLN). The electronic device may remove cells on the surface based on the first cutting line (CLN). For example, the electronic device may remove cells through which the first cutting line (CLN) passes on the surface.

In some embodiments, the electronic device may divide the cells through which the first cutting line (CLN) passes on the surface into sub-cells. That is, the electronic device may perform subdivision on the cells through which the first cutting line (CLN) passes. The electronic device may perform image processing on the divided sub-cells.

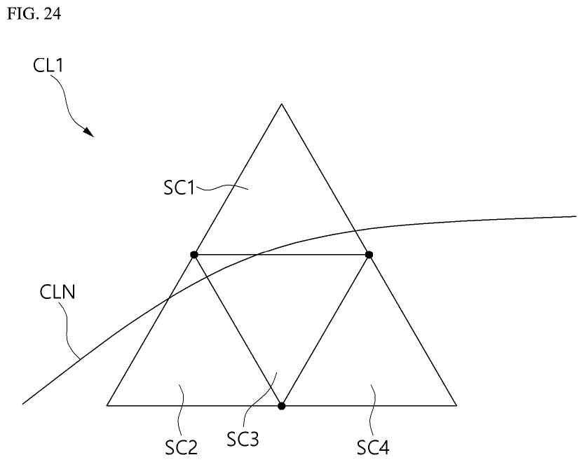

Referring to FIG. 24, the electronic device according to an embodiment may divide a cell (CL1) through which the first cutting line (CLN) passes on the surface of the vessel network to generate sub-cells (SC1 to SC4). For example, the electronic device may generate sub-cells (SC1 to SC4) by connecting the midpoints of each edge of the cell (CL1). However, the embodiment is not limited thereto, and the electronic device may divide the cell (CL1) in various ways. For example, the electronic device may divide the cell (CL1) based on the points where the cell (CL1) intersects with the first cutting line (CLN).

The electronic device may remove the sub-cells (SC1 to SC3) among the sub-cells (SC1 to SC4) through which the first cutting line (CLN) passes. That is, the electronic device may retain only sub-cell (SC4) in the cell (CL1) and remove the sub-cells (SC1 to SC3).

In this way, as the electronic device generates the sub-cells (SC1 to SC4), it may increase the surface density of the vessel network and minimize damages to the original surface. This is because removing the sub-cells (SC1 to SC3) through which the first cutting line (CLN) passes after subdivision results in less data loss compared to removing the entire cell (CL1) (i.e., all the sub-cells (SC1 to SC4)).

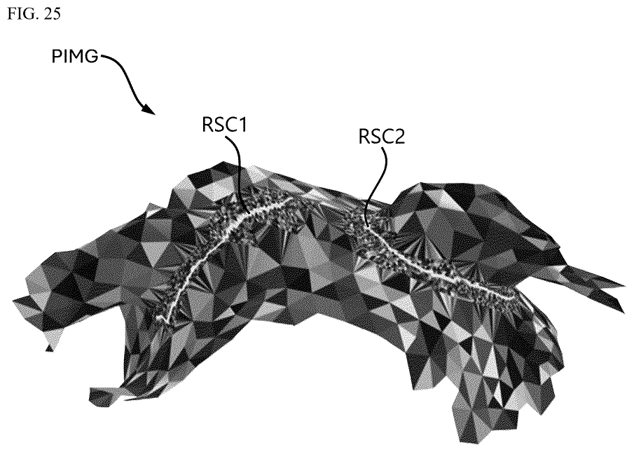

The result of the electronic device removing sub-cells through which the first cutting line (CLN) passes and sub-cells through which the second cutting line passes on the surface may be as shown in FIG. 25.

Referring to FIG. 25, it can be seen that cells on the surface of the vessel network through which the first and second cutting lines pass are divided into sub-cells. The electronic device may generate an image (PIMG) by removing the first sub-cells through which the first cutting line passes and the second sub-cells through which the second cutting line passes. For example, the part where the first sub-cells are removed may be a region (RSC1), and the part where the second sub-cells are removed may be a region (RSC2).

The electronic device may generate a new mesh image on the remaining surface where the first and second sub-cells have been removed. The surface from which the first sub-cells were removed may leave a first open vessel image and a second open vessel image, and the surface from which the second sub-cells were removed may leave a third open vessel image and a fourth open vessel image. In other words, the first and second open vessel images may be generated based on the region (RSC1), and the third and fourth open vessel images may be generated based on the region (RSC2). The first to fourth open vessel images may each have a boundary.

The electronic device may close the boundaries of the first to fourth mesh images. The configuration of closing the boundaries by the electronic device can be understood as an operation to fill the blood vessels, which are open due to removal, with cells. The electronic device may perform a remesh algorithm on the mesh images with closed boundaries to generate a remesh image.

The electronic device may add the remesh image to the image from which the sub-cells were removed (for example, the image (PIMG) in FIG. 25). The electronic device may perform the image addition based on information of the cells (or sub-cells) neighboring the removed sub-cells. If the electronic device connects the remesh image with the image (PIMG), the electronic device may remove an image where a region is separated.

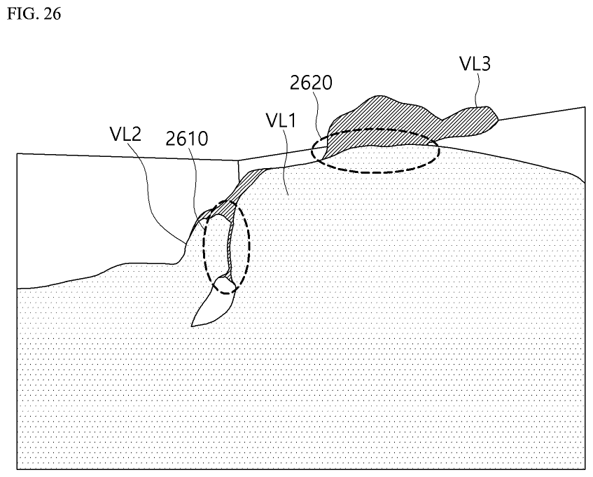

The final image generated by the electronic device after separating the kissing vessels may be as shown in FIG. 26. Referring to FIG. 26, the electronic device has separated the second kissing vessel (VL2) and the third kissing vessel (VL3) from the first kissing vessel (VL1). With respect to contact areas 2610, 2620 of the kissing vessels (VL1 to VL3), it can be confirmed that the first kissing vessel (VL1) has been completely separated from the second kissing vessel (VL2) and the third kissing vessel (VL3). In other words, as the electronic device removes noises that may occur during the imaging process, the clarity and accuracy can be improved, enabling precise analysis of the first kissing vessel (VL1). Additionally, it reduces the need for re-imaging, shortens treatment time, and can reduce risks during a (medical) procedure or surgery, thereby enhancing reliability.

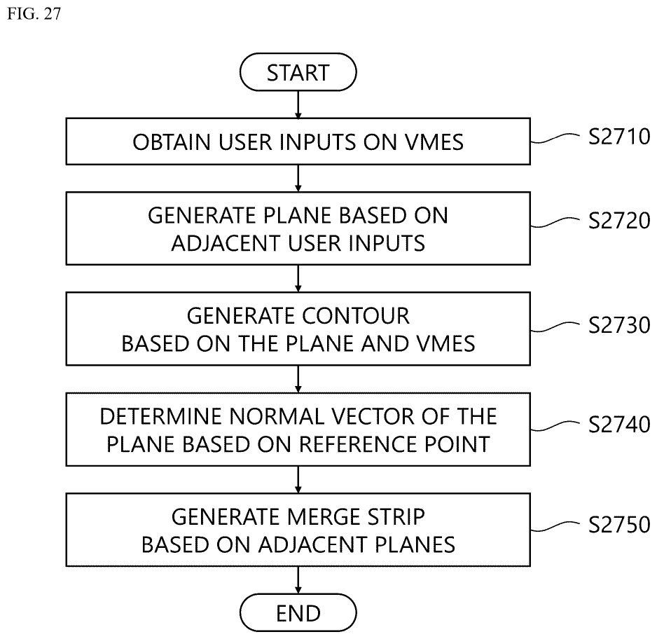

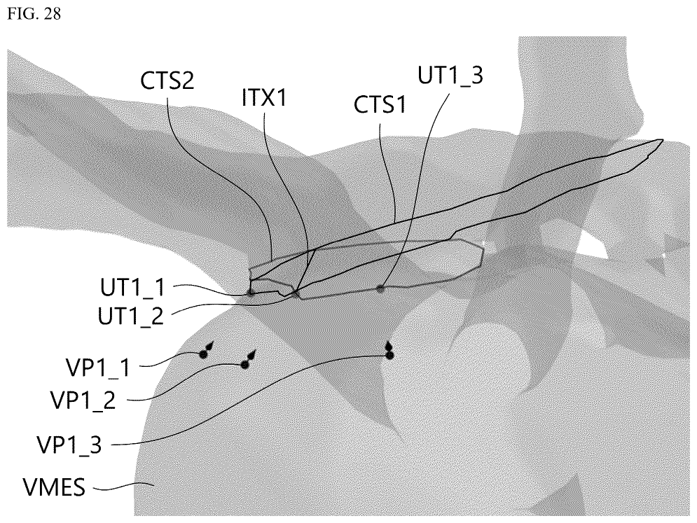

FIG. 27 illustrates a flowchart of a method for generating a merge strip by an electronic device according to an embodiment, and FIGS. 28 to 35 illustrate the process in detail.

Referring to FIGS. 27 and 28, an electronic device according to an embodiment may obtain user inputs (UT1_1 to UT1_3) on a vessel mesh (VMES) (S2710). For example, the electronic device may display the vessel mesh (VMES) from a specific camera viewpoint on a display. The electronic device may adjust the camera view by responding to a user's command to change the viewpoint and display the vessel mesh (VMES) from the updated viewpoint.

The electronic device may display user inputs (UT1_1 to UT1_3) corresponding to camera viewpoints (VP1_1 to VP1_3) and input commands. For example, the user may provide an input command from a camera viewpoint (VP1_1), and the electronic device may display the user input (UT1_1) in response to the viewpoint and the input command. The electronic device may generate a straight line in 3D space connecting the camera viewpoint (VP1_1) and the input command and may represent a first point of contact with the vessel mesh (VMES) as the user input (UT1_1). Similarly, the electronic device may display additional user inputs (UT1_2, UT1_3). Vectors pointing from the camera viewpoints (VP1_1 to VP1_3) to the user inputs (UT1_1 to UT1_3) may be shown as arrows.

For convenience of explanation, FIG. 28 illustrates different camera viewpoints (VP1_1 to VP1_3) corresponding to the user inputs (UT1_1 to UT1_3). However, the embodiments are not limited thereto, and multiple user inputs may correspond to the same camera viewpoint. In other words, in some embodiments, the user may either maintain or change the camera viewpoint while providing inputs to the electronic device.

The electronic device may generate a plane based on adjacent user inputs (S2720). The electronic device may use camera viewpoints corresponding to the user inputs. For example, the electronic device may generate a first plane based on adjacent user inputs (UT1_1, UT1_2) and camera viewpoints (VP1_1, VP1_2). Further, the electronic device may generate a second plane based on user inputs (UT1_2, UT1_3) and camera viewpoints (VP1_2, VP1_3).

The electronic device may generate a contour based on the planes and the vessel mesh (VMES) (S2730). For example, the electronic device may generate a contour (CTS1) where the first plane intersects the vessel mesh (VMES). Likewise, the electronic device may generate a contour (CTS2) where the second plane intersects the vessel mesh (VMES). The electronic device may represent an intersection line (ITX1) of the first and second planes. The electronic device may generate a merge strip using the intersection line (ITX1), as will be described later.

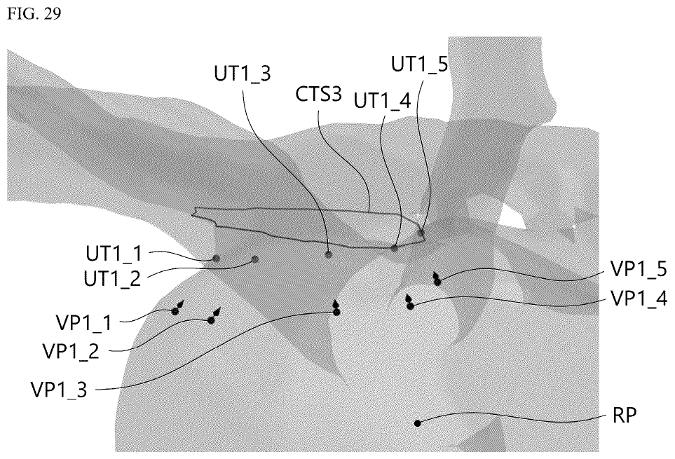

The electronic device may generate planes and contours for all user inputs. Referring to FIG. 29, the electronic device may obtain user inputs (UT1_1 to UT1_5) from camera viewpoints (VP1_1 to VP1_5). The electronic device may generate a contour (CTS3) corresponding to user inputs (UT1_4, UT1_5). Although not shown, the electronic device may also generate a contour for user inputs (UT1_3, UT1_4).

The electronic device may determine a normal vector of the plane based on a reference point (RP) (S2740). The electronic device may select one normal vector pointing opposite to the reference point (RP) among two possible normal vectors of the plane. That is, if the reference point (RP) is located on one side of the plane, the electronic device may select a normal vector pointing to the other side. In some embodiments, the electronic device may use dot product of the normal vector and the reference point (RP) for the determination.

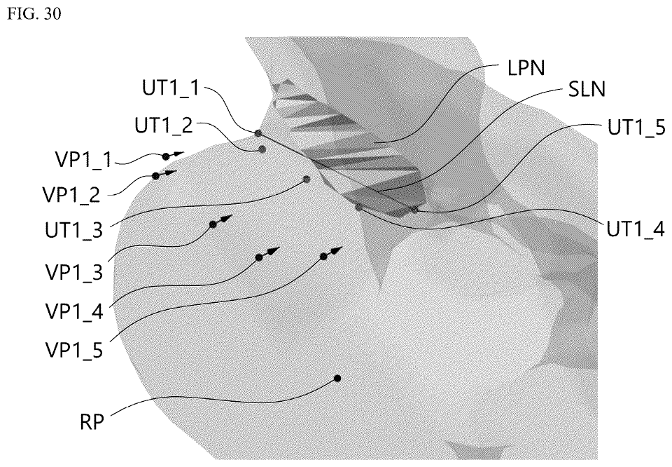

Referring to FIG. 30, the electronic device may generate a plane (LPN) based on user inputs (UT1_4, UT1_5) and camera viewpoints (VP1_4, VP1_5). The electronic device may determine an arbitrary point located on one side of the plane (LPN) as the reference point (RP).

The electronic device may generate a line segment (SLN) based on the first user input (UT1_1) and the last user input (UT1_5). The electronic device may calculate perpendicular distances from the line segment (SLN) to each user input (UT1_1 to UT1_5). The electronic device may determine the reference point (RP) based on the user input with the greatest distance (e.g., UT1_3) and the plane (LPN). For example, the electronic device may generate the reference point (RP) in a direction opposite to the user input with the greatest distance relative to the plane (LPN). That is, the reference point (RP) may be on one side of the plane (LPN), while the user input with the greatest distance is on the other side.

In some embodiments, the electronic device may determine an aneurysm center point as the reference point (RP). The aneurysm center point may represent a point characterizing the aneurysm region and may be determined either through user input or automatically by the electronic device. In some embodiments, an artificial intelligence model trained to output the aneurysm center point may be used.

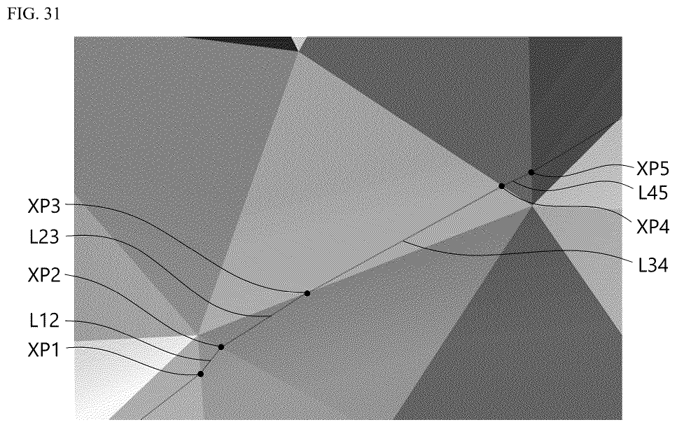

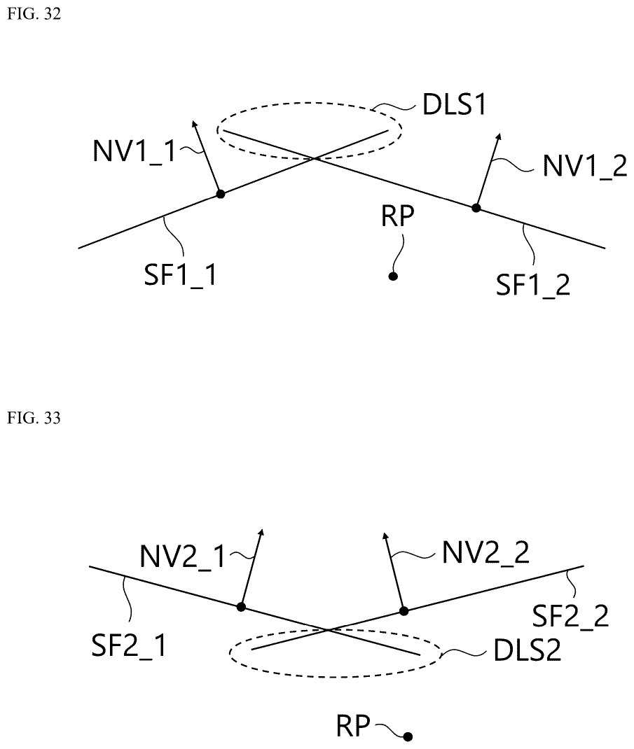

The electronic device may generate a merge strip based on adjacent planes (S2750). Referring to FIG. 31, the planes generated by the electronic device according to an embodiment may pass through cells (illustrated as a plurality of triangles in FIG. 31) of the vessel mesh (VMES). The electronic device may generate intersection points (XP1 to XP5) between the planes and the cells. The intersection points (XP1 to XP5) may be located at edges of the cells.

The electronic device may convert lines (L12, L23, L34, and L45) between the intersection points (XP1 to XP5) into polygons and generate a 3D surface based on the converted polygons. The electronic device may generate 3D surfaces from adjacent planes. The electronic device may remove portions of the adjacent 3D surfaces based on the reference point (RP).

Referring to FIG. 32, the electronic device may generate 3D surfaces (SF1_1 and SF1_2) from adjacent planes. The electronic device may remove an overlapping section (DLS1) (or intersection portion) based on the reference point (RP). For example, the electronic device may use a normal vector (NV1_1) of the surface (SF1_1) and a normal vectors (NV1_2) of the surface (SF1_2). The normal vector (NV1_1) may originate from a center point (SCP1_1) of the surface (SF1_1), and the normal vector (NV1_2) may originate from a center point (SCP1_2) of surface (SF1_2). In some embodiments, the electronic device may determine the midpoint of user inputs (UT1_1 and UT1_2) as the center point (SCP1_1) and the midpoint of user inputs (UT1_2 and UT1_3) as the center point (SCP1_2). The normal vectors (NV1_1 and NV1_2) correspond to the normal vectors of the adjacent planes, and the details described with reference to FIG. 29 apply here.

The electronic device may set the surface (SF1_1), where the user input is prioritized, as a reference among surfaces (SF1_1 and SF1_2). In some embodiments, the electronic device may generate a vector (DV1) corresponding to the difference between the normal vector (NV1_1) and the normal vector (NV1_2). In some embodiments, the electronic device may generate a vector (DV1) directed from the center point (SCP1_1) of surface (SF1_1) to the center point (SCP1_2). The vector (DV1) may be a difference vector of the normal vector (NV1_1) and the normal vector (NV1_2).

The electronic device may calculate the dot product of the normal vector (NV1_1) and the vector (DV1) and remove the upper overlapping section (DLS1) if the dot product value is less than 0. Adjacent planes may have an intersection line (e.g., ITX1 in FIG. 28), and the electronic device may remove the overlapping section (DLS1) in one direction based on the intersection line.

Referring to FIG. 33, the electronic device may generate 3D surfaces (SF2_1 and SF2_2) from adjacent planes. The electronic device may remove an overlapping section (DLS2) based on the reference point (RP). For example, the electronic device may use a normal vector (NV2_1) of surface (SF2_1) and a normal vector (NV2_2) of the surface (SF2_2). The normal vector (NV2_1) may originate from a center point (SCP2_1) of surface (SF2_1), and the normal vector (NV2_2) may originate from the center point (SCP2_2) of surface (SF2_2).

The electronic device may set the surface (SF2_1), where the user input is prioritized, as a reference among surfaces (SF2_1 and SF2_2). In some embodiments, the electronic device may generate a vector (DV2) corresponding to the difference between the normal vector (NV2_1) and the normal vector (NV2_2). In some embodiments, the electronic device may generate a vector (DV2) directed from the center point (SCP2_1) of surface (SF2_1) to the center point (SCP2_2). The vector (DV2) may be a difference vector of the normal vector (NV2_1) and the normal vector (NV2_2).

The electronic device may calculate the dot product of the normal vector (NV2_1) and the vector (DV2) and remove the lower overlapping section (DLS2) if the dot product value is greater than 0. Adjacent planes may have an intersection line, and the electronic device may remove the overlapping section (DLS2) in one direction based on the intersection line.

In FIGS. 32 and 33, for convenience of explanation, the surface (SF1_1 or SF2_1) where the user input is prioritized is set as the reference, but the embodiment is not limited thereto. For example, the electronic device may set the surface (SF1_2 or SF2_2), where the user input follows, as the reference and use the inverse vector of the vector (DV1 or DV2) for the dot product.

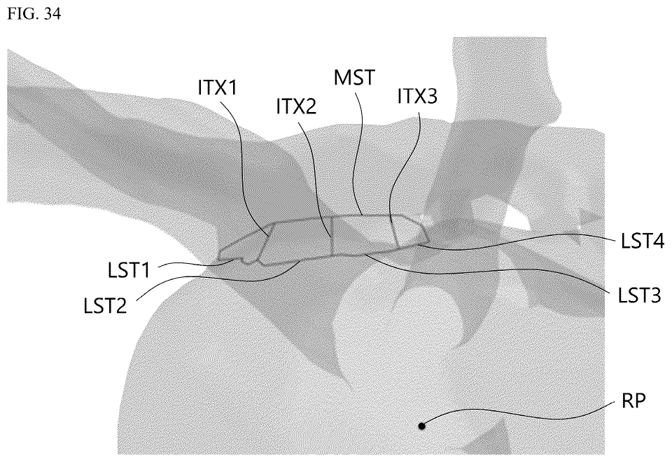

The electronic device may connect the remaining surfaces that are not removed to generate a merge strip. The electronic device may connect line strips of each surface. Referring to FIG. 34, the electronic device may generate line strips (LST1 and LST2) based on the intersection line (ITX1) of the first plane and the second plane. Similarly, the electronic device may generate line strips (LST3 and LST4) based on intersection lines (ITX2, ITX3). The electronic device may connect the line strips (LST1 to LST4) to generate a merge strip (MST).

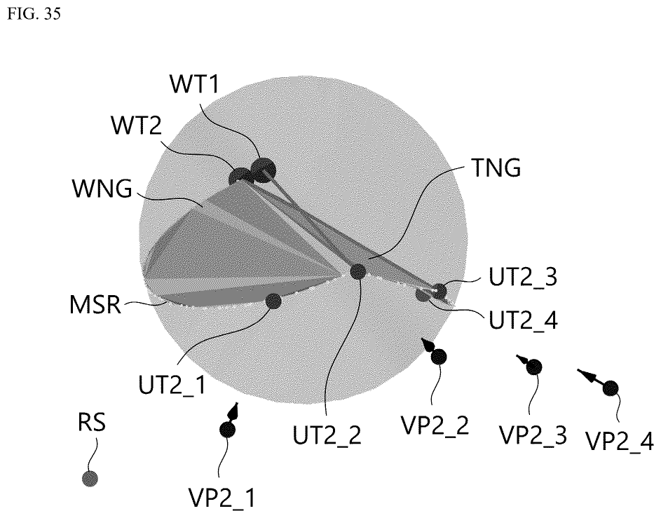

Referring to FIG. 35, an electronic device according to an embodiment may generate line strips based on camera viewpoints (VP2_1 to VP2_4), user inputs (UT2_1 to UT2_4), and a reference point (RS) and connect the line strips to generate a merge strip (MSR). The electronic device may convert polygons generated by connecting the line strips into a surface. For example, the electronic device may generate polygons of types such as wing, triangle, or rectangle.

The electronic device may determine the type of polygon based on whether the polygons of adjacent user inputs intersect. For example, if the polygons of the adjacent user inputs (e.g., UT2_1 and UT2_2) do not intersect, the electronic device may generate a wing-type polygon (WNG).

If the polygons of the adjacent user inputs (e.g., UT2_2 and UT2_3) intersect, the electronic device may generate a triangle-type polygon (TNG). A first line (or vector) generated based on the user input (UT2_2) and the camera viewpoint (VP2_2) may intersect the vessel mesh secondly at a point (WT1).

A second line (or vector) generated based on the user input (UT2_3) and the camera viewpoint (VP2_3) may also intersect the vessel mesh secondly at a point (WT2). At this time, the second line may pass through the polygon generated by the first line. Thus, the electronic device may generate a triangle-type polygon based on the user inputs (UT2_2 and UT2_3) and the point (WT2). The electronic device may separate adhered vessels using the merge strip (MSR) generated as described above.

Even when noise occurs due to a threshold in the region of interest (e.g., an aneurysm region) causing vessels to adhere, the electronic device enables vessel separation based on user input, facilitating more precise measurement and analysis of the aneurysm.

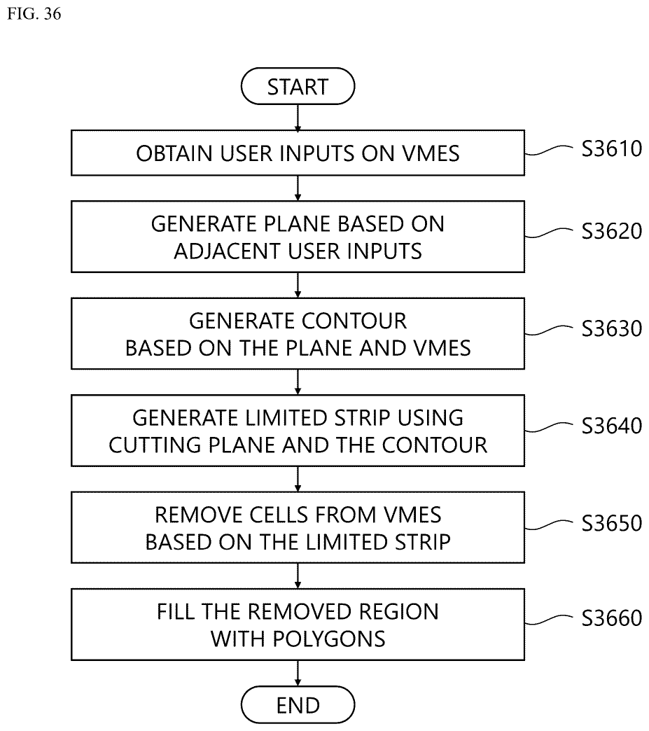

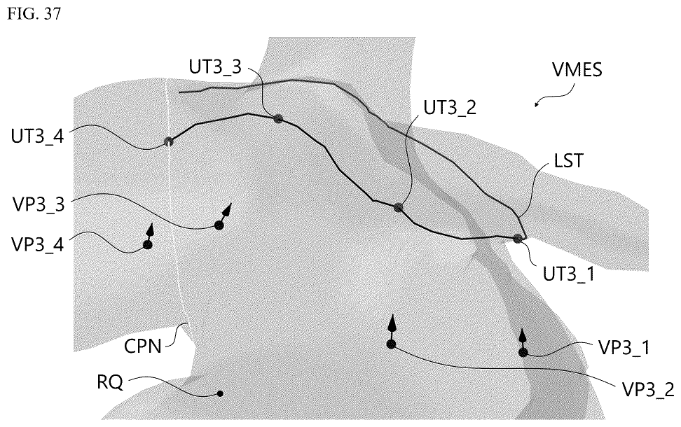

FIG. 36 illustrates a flowchart of a method for separating adhered vessels by an electronic device according to an embodiment, and FIGS. 37 to 44 illustrate a method for separating adhered vessels by an electronic device according to an embodiment. Here, adhered vessels may refer to kissing vessels or contacting vessels.

Referring to FIGS. 36 and 37, an electronic device according to an embodiment may obtain user inputs (UT3_1 to UT3_4) on the vessel mesh (VMES) (S3610). The method for generating the vessel mesh (VMES) described earlier may apply here. The electronic device may generate a plane based on adjacent user inputs (S3620). The electronic device may use camera viewpoints (VP3_1 to VP3_4) corresponding to the user inputs (UT3_1 to UT3_4). The electronic device may generate the contour based on the plane and the vessel mesh (VMES) (S3630). The steps in FIG. 36 (S3610, S3620, and S3630) are analogous to the steps described in FIG. 27 (S2710, S2720, and S2730). Thus, redundant explanations are omitted.

The electronic device may generate a limited strip (LST) (or a finite strip) using the contour and a cutting plane (CPN) (S3640). The electronic device may generate the cutting plane (CPN) based on the last user input (UT3_4), the camera viewpoint (VP3_4) corresponding to the user input (UT3_4), and a reference point (RQ). The reference point (RQ) may be generated using the same method as the reference point (RP) in FIG. 29. The electronic device may generate the limited strip (LST) by removing portions of the contour based on the cutting plane (CPN). For example, the electronic device may retain the portions of the contour where the user inputs (UT3_1 to UT3_4) exist and remove the portions where the user inputs (UT3_1 to UT3_4) do not exist.

The electronic device may remove cells from the vessel mesh (VMES) based on the limited strip (LST) (S3650). The electronic device may remove the cells in the vessel mesh (VMES) that the limited strip (LST) intersects. In some embodiments, the electronic device may divide the intersected cells into sub-cells and remove at least one sub-cell.

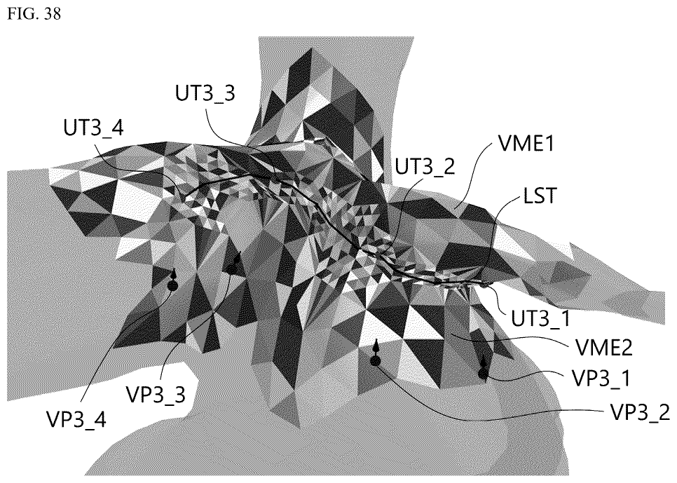

Referring to FIG. 38, it may be observed that, in the vessel mesh (VMES) according to an embodiment, a first vessel (VME1) and a second vessel (VME2) are adhered. The electronic device may separate the first vessel (VME1) and the second vessel (VME2).

An electronic device according to an embodiment may divide cells intersected by the limited strip (LST) in the vessel mesh (VMES) into sub-cells. The electronic device may divide the cells based on a cell division level. For example, smaller and more sub-cells are generated for a higher cell division level, while larger and fewer sub-cells are generated for a lower division level. The explanation of cells and sub-cells in FIG. 24 may apply here, and thus, redundant details are omitted.

The electronic device may remove sub-cells based on a cell removal level. For example, more sub-cells (or cells) are removed for a higher cell removal level, while fewer sub-cells are removed for a lower removal level. Neighboring sub-cells around the sub-cells intersected by the limited strip (LST) may also be removed for a higher cell removal level. In some embodiments, the cell division level and the cell removal level may be configured by a user.

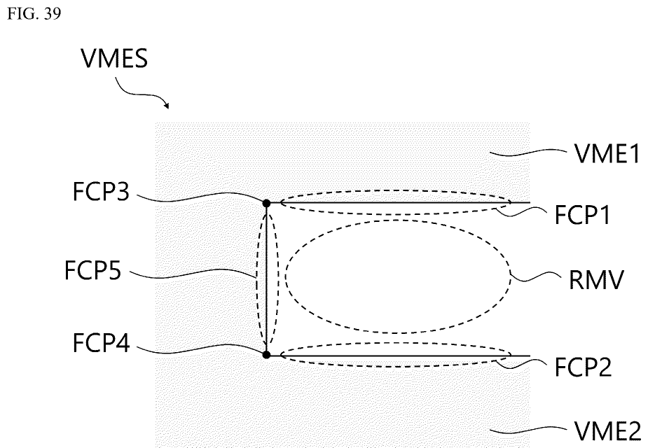

The electronic device may fill cell removed areas with polygons (S3660). Referring to FIG. 39, sub-cells (or cells) may be removed from the vessel mesh (VMES) according to an embodiment. Consequently, an open area (RMV) is created in the 3D space, and although the first vessel (VME1) and the second vessel (VME2) in the vessel mesh (VMES) are separated, the first and second vessels (VME1 and VME2) may not be closed but remain open. For example, the lower region (FCP1) of the first vessel (VME1), the upper region (FCP2) of the second vessel (VME2), corner regions (FCP3 and FCP4), and connection region (FCP5) between the first vessel (VME1) and the second vessel (VME2) may remain open. The electronic device may fill the regions (FCP1 to FCP5) with polygons. The regions (FCP1 to FCP5) represent boundary regions of the vessel mesh (VMES) and may include a plurality of edges.

The electronic device may use various methods to fill the regions (FCP1 to FCP5). The electronic device according to an embodiment may fill regions (FCP1 and FCP2) by generating polygons using the edges of the boundary regions, user inputs, and camera viewpoints.

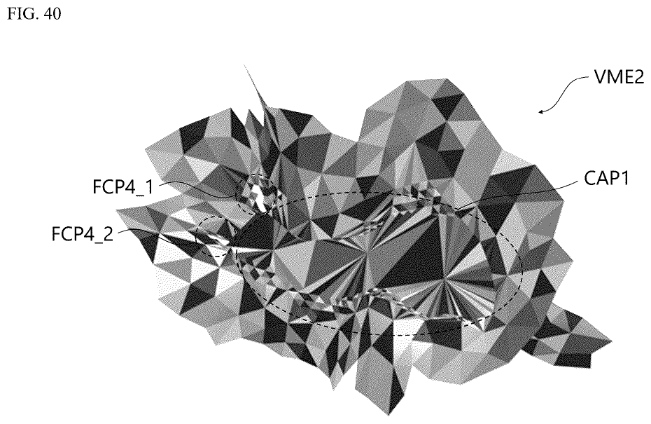

FIG. 40 illustrates a configuration for filling the region (FCP2) of the second vessel (VME2) by an electronic device according to an embodiment. Referring to FIG. 40, the electronic device may fill the region (FCP2) by generating polygons using the edges of the region (FCP2), user inputs, and camera viewpoints. The same descriptions for the user inputs and the camera viewpoints described in FIG. 37 may apply here. The electronic device may generate polygons using methods described with reference to FIGS. 27 to 35. The electronic device may generate polygons such as wings, triangles, or rectangles. The electronic device may generate a closed region (CAP1) by filling the upper region (FCP2) of the second vessel (VME2) with polygons. It may be observed that regions (FCP4_1 and FCP4_2) corresponding to the corner region (FCP4) remain unfilled and open. The electronic device may generate polygons for region (FCP1) using the same method.

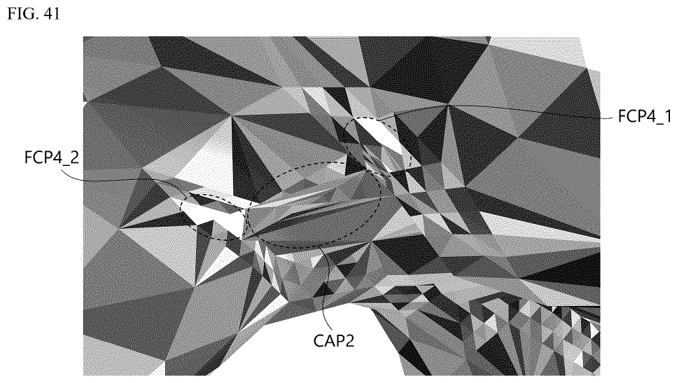

FIG. 41 illustrates a configuration for filling the region (FCP5) by an electronic device according to an embodiment. Referring to FIG. 41, the electronic device may generate polygons using two endpoints of the corner region (FCP3) and two endpoints of the corner region (FCP4). The two endpoints of the corner region (FCP4) may refer to an endpoint of the region (FCP4_1) and an endpoint of the region (FCP4_2). The electronic device may generate a closed region (CAP2) by filling the region (FCP5) with polygons. It may be observed that regions (FCP4_1 and FCP4_2) remain unfilled and open.

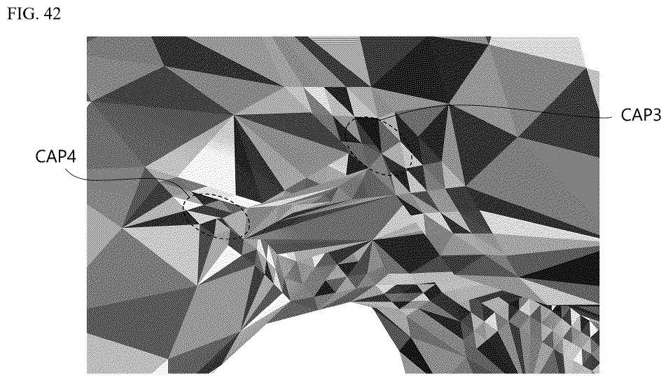

FIG. 42 illustrates a configuration for filling the region (FCP4) by an electronic device according to an embodiment. Referring to FIG. 42, the electronic device may generate polygons using the last user input (e.g., UT3_4 in FIG. 37), camera viewpoint (e.g., VP3_4 in FIG. 37) corresponding to the last user input, and two endpoints of the limited strip (e.g., LST in FIG. 37) generated by the cutting plane (e.g., CPN in FIG. 37). The two endpoints of the limited strip may refer to the points where the limited strip intersects the cutting plane. The electronic device may generate closed regions (CAP3 and CAP4) by filling the regions (FCP4_1 and FCP4_2) with polygons. The same method may be used to generate polygons for the region (FCP3).

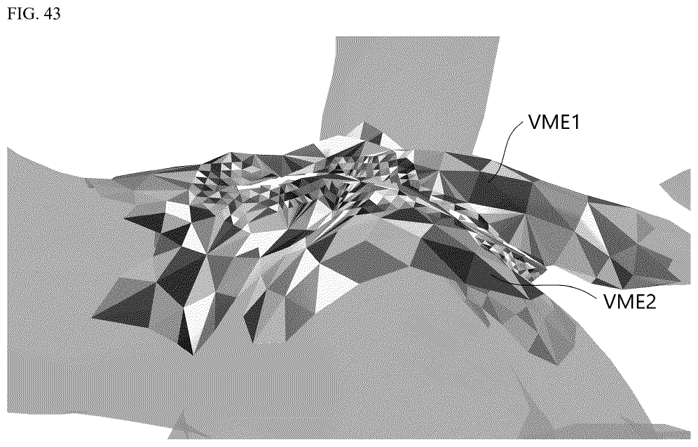

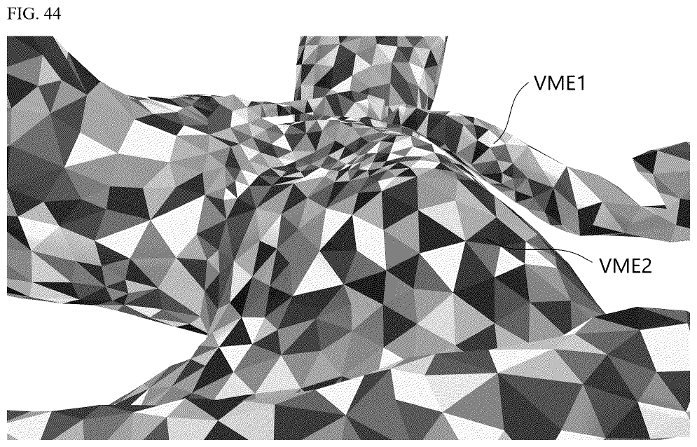

Through the process, the electronic device may separate two vessels (VME1 and VME2) that were previously represented adhered into respective vessels, as shown in FIG. 43. The electronic device may perform a remesh algorithm on the vessel mesh (VMES) with the separated vessels (VME1 and VME2). The result of the remesh algorithm performed on the vessel mesh (VMES) is shown in FIG. 44.

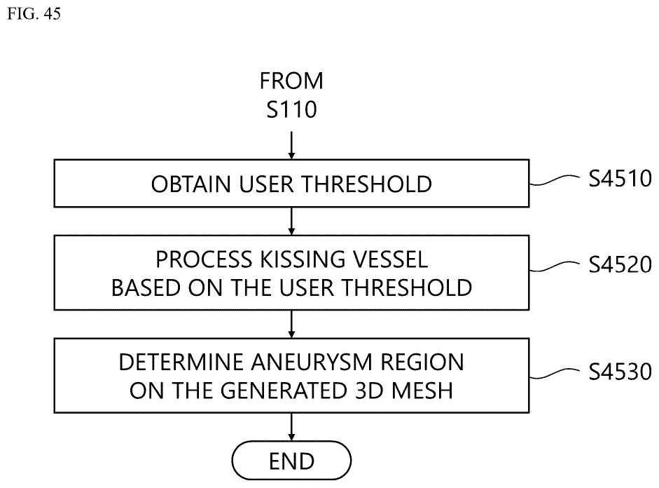

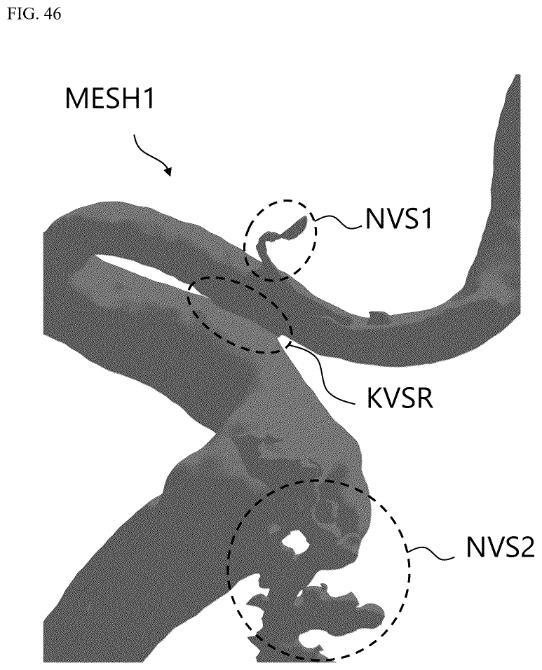

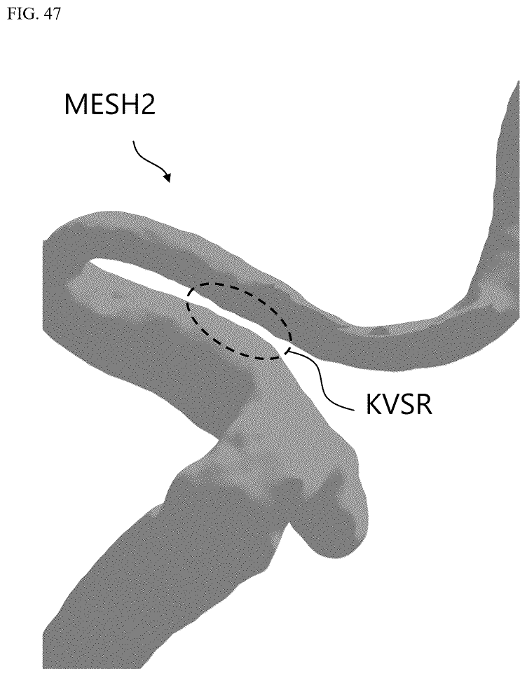

FIG. 45 illustrates a flowchart of a method for separating adhered vessels by an electronic device according to an embodiment, and FIGS. 46 and 47 illustrate a method for separating adhered vessels by an electronic device according to an embodiment.

Referring to FIG. 45, an electronic device according to an embodiment may obtain input images and generate a vessel mesh (S110). The electronic device may use an initial threshold calculated by an algorithm based on the input images to generate a 3D vessel mesh. The electronic device may classify voxels in the input images using the initial threshold. For example, the electronic device may classify voxels with values greater than the initial threshold as vessels and voxels with values equal to or less than the initial threshold as background. Referring to FIG. 46, the electronic device may generate a vessel mesh (MESH1) based on the initial threshold. The vessel mesh (MESH1) may include an adhered vessel region (KVSR) and noise regions (NVS1 and NVS2). The adhered vessel region (KVSR) represent vessels that are actually separate but appear adhered and the noise regions (NVS1 and NVS2) represent areas that are not vessels but may have been generated due to noise from contrast agent or imaging process. The description of step S110 applies as described with reference to FIG. 1, and redundant details are omitted.

The electronic device may obtain a user threshold (S4510). The user may input the user threshold by adjusting a threshold on the electronic device. For example, the user may modify the threshold via a display interface. Consequently, the threshold may be changed from the initial threshold to the user threshold.

The electronic device may process the adhered vessels based on the user threshold (S4520). The electronic device may process the adhered vessels according to changes in the user threshold. That is, the user may observe changes in the vessel representation in real time corresponding to the changed user threshold and set the threshold accordingly. Referring to FIG. 47, the electronic device may generate a vessel mesh (MESH2) based on the user threshold. It may be observed that the adhered vessel region (KVSR) in vessel mesh (MESH1) have been separated in vessel mesh (MESH2). Further, noise regions are also removed in vessel mesh (MESH2).

In some embodiments, the electronic device may detect a threshold at which the adhered vessels begin to separate and propose the threshold to the user. In a configuration where the electronic device proposes the threshold, the process of obtaining the user threshold may be omitted. The user may fine-tune the user threshold based on the proposed threshold. In other words, the electronic device may propose a minimal threshold that outputs the largest vessel mesh without causing adhered vessels or noise.

The electronic device may determine an aneurysm region from the generated 3D mesh (S4530). The generated 3D mesh refers to the mesh generated by adjusting the threshold. The configuration for determining the aneurysm region by the electronic device has already been described with reference to networks (e.g., vessel network, sticky network, max-cut network, flow network, aneurysm network, etc.), and redundant explanations are omitted.

While this invention has been described in connection with what is presently considered to be practical embodiments, it is to be understood that the invention is not limited to the disclosed embodiments. On the contrary, it is intended to cover various modifications and equivalent arrangements included within the spirit and scope of the appended claims.

In some embodiments, each component or a combination of two or more components described with reference to FIG. 1 to FIG. 47 may be implemented with digital electronic and/or optical circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” and “computer-readable medium” refer to any computer program product, non-transitory computer readable medium, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

The processes and logic flows described in this specification can be performed by one or more programmable processors, also referred to as data processing hardware, executing one or more computer programs to perform functions by operating on input data and generating output. The processes and logic flows can also be performed by special purpose logic circuitry, e.g., an FPGA (field programmable gate array) or an ASIC (application specific integrated circuit). Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, one or more aspects of the disclosure can be implemented on a computer having a display device, e.g., a CRT (cathode ray tube), LCD (liquid crystal display) monitor, LED (light-emitting diode) monitor, OLED (organic LED) monitor, or touch screen for displaying information to the user and optionally a keyboard and a pointing device, e.g., a mouse or a trackball, by which the user can provide input to the computer. Other kinds of devices can be used to provide interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

A number of implementations have been described. Nevertheless, it will be understood that various modifications may be made without departing from the spirit and scope of the disclosure. Accordingly, other implementations are within the scope of the following claims.

What is claimed is:

1. A method for generating an aneurysm region performed by an electronic device, comprising:
obtaining, by at least one processor, input images;
generating, by the at least one processor, a vessel mesh based on the input images;
determining, by the at least one processor, an adhered vessel from the vessel mesh; and
separating, by the at least one processor, the adhered vessel based on user input.

2. The method of claim 1, wherein the separating the adhered vessel based on user input comprises:
obtaining a plurality of user inputs from a plurality of camera viewpoints;
generating a merge strip based on the plurality of camera viewpoints and the plurality of user inputs; and
separating the adhered vessel based on the merge strip.

3. The method of claim 2, wherein the obtaining a plurality of user inputs from a plurality of camera viewpoints comprises:
obtaining a first user input from a first camera viewpoint;
obtaining a second user input from a second camera viewpoint; and
obtaining a third user input from a third camera viewpoint; and
wherein the generating a merge strip based on the plurality of camera viewpoints and the plurality of user inputs comprises:
generating a first plane based on the first camera viewpoint, the second camera viewpoint, the first user input, and the second user input;
generating a second plane based on the second camera viewpoint, the third camera viewpoint, the second user input, and the third user input; and
generating the merge strip based on the first plane and the second plane.

4. The method of claim 3, wherein the generating the merge strip based on the first plane and the second plane comprises:
generating a first contour where the first plane intersects the vessel mesh;
generating a second contour where the second plane intersects the vessel mesh; and
generating the merge strip based on the first contour and the second contour.

5. The method of claim 4, wherein the generating the merge strip based on the first contour and the second contour comprises:
determining a first normal vector of the first plane and a second normal vector of the second plane based on a reference point; and
removing an intersection portion from the first contour and the second contour based on the first normal vector and the second normal vector.

6. The method of claim 5, wherein the removing the intersection portion from the first contour and the second contour based on the first normal vector and the second normal vector comprises:
generating a difference vector between the first normal vector and the second normal vector; and
removing the intersection portion from the first contour and the second contour based on a dot product value of the first normal vector and the difference vector.

7. The method of claim 5, wherein the generating the merge strip based on the first contour and the second contour further comprises:
generating a third plane based on the last two inputs among the plurality of user inputs;
generating a line segment based on the first input and the last input among the plurality of user inputs;
calculating a plurality of distances from the plurality of user inputs to the line segment; and
determining the reference point based on the third plane and a user input corresponding to the longest distance among the plurality of distances.

8. The method of claim 2, wherein the separating the adhered vessel based on the merge strip comprises:
determining cells through which the merge strip passes in the vessel mesh;
dividing the determined cells into sub-cells; and
removing sub-cells through which the merge strip passes among the divided sub-cells.

9. The method of claim 2, wherein the separating the adhered vessel based on the merge strip comprises:
determining a cutting plane;
generating a limited strip based on the merge strip and the cutting plane; and
separating the adhered vessel based on the limited strip.

10. The method of claim 9, wherein the determining the cutting plane comprises:
determining the cutting plane based on the last input among the plurality of user inputs, the last camera viewpoint corresponding to the last input, and a reference point.

11. The method of claim 10, wherein the determining the cutting plane further comprises:
generating a plane based on the last two inputs among the plurality of user inputs;
generating a line segment based on the first input and the last input among the plurality of user inputs;
calculating a plurality of distances from the plurality of user inputs to the line segment; and
determining the reference point based on the generated plane and a user input corresponding to the longest distance among the plurality of distances.

12. The method of claim 9, wherein the separating the adhered vessel based on the limited strip comprises:
determining cells through which the limited strip passes in the vessel mesh;
dividing the determined cells into sub-cells; and
removing sub-cells through which the limited strip passes among the divided sub-cells.

13. The method of claim 12, wherein the adhered vessel comprises a first vessel and a second vessel, and the method further comprises:
filling a boundary region of the first vessel, a boundary region of the second vessel, a connection region of the first vessel and the second vessel, a corner region of the first vessel, and a corner region of the second vessel with polygons.

14. The method of claim 13, wherein the filling with polygons comprises:
generating first polygons based on edges of the boundary region of the first vessel, the plurality of camera viewpoints, and the plurality of user inputs; and
generating second polygons based on edges of the boundary region of the second vessel, the plurality of camera viewpoints, and the plurality of user inputs.

15. The method of claim 13, wherein the filling with polygons comprises:
generating polygons based on two endpoints of the corner region of the first vessel and two endpoints of the corner region of the second vessel.

16. The method of claim 13, wherein the filling with polygons comprises:
generating polygons based on the last input among the plurality of user inputs, the last camera viewpoint corresponding to the last input, and two endpoints of the limited strip.

17. The method of claim 13, further comprising:
performing a remesh algorithm on the vessel mesh comprising the separated first vessel and second vessel.

18. An electronic device comprising:
a processor and a memory connected to the processor,
wherein the memory is configured to store a program,
wherein the processor is configured to execute the program, and
when the program is executed, the steps of the method according to claim 1 are implemented.
